SEQUENCE LISTING (38 sequences)

SEQ ID NO: 1 - protein, 26 aa, Staphylococcus carnosus
IRSATEIWWLPNLTADQKWAFIYKLV

SEQ ID NO: 2 - protein, 29 aa, Staphylococcus carnosus
EMWIAWEEIRDLPNLNGWQMTAFIASLLD

SEQ ID NO: 3 - protein, 36 aa, Staphylococcus carnosus
VDNKFNKEMWIAWEEIRDLPNLNGWQMTAFIASLLD

SEQ ID NO: 4 - protein, 51 aa, Staphylococcus carnosus
EMWIAWEEIRDLPNLNGWQMTAFIASLLDDPSQSANLLAEAKKLNDAQAPK

SEQ ID NO: 5 - protein, 58 aa, Staphylococcus carnosus
VDNKFNKEMWIAWEEIRDLPNLNGWQMTAFIASLLDDPSQSANLLAEAKKLNDAQAPK

SEQ ID NO: 6 - protein, 46 aa, Staphylococcus carnosus
LAEAKVLANRELDKYGVSDFYKRLINKAKTVEGVEALKLHILAALP

SEQ ID NO: 7 - protein, 46 aa, Staphylococcus carnosus
LAEAKEAANAELDSYGVSDFYKRLIDKAKTVEGVEALKDAILAALP

SEQ ID NO: 8 - protein, 47 aa, Staphylococcus carnosus
GLAEAKEAANAELDSYGVSDFYKRLIDKAKTVEGVEALKDAILAALP

SEQ ID NO: 9 - protein, 46 aa, Staphylococcus carnosus
LAEAKVLANRELDKYGVSDYYKNLINNAKTVEGVKALIDEILAALP

SEQ ID NO: 10 - protein, 46 aa, Staphylococcus carnosus
LAEAKVLALRELDKYGVSDYYKDLIDKAKTVEGVKALIDEILAALP

SEQ ID NO: 11 - protein, 4 aa, Staphylococcus carnosus
GFLG

SEQ ID NO: 12 - protein, 215 aa, Staphylococcus carnosus
HEHEHEVDAKYAKEIRSATEIWWLPNLTADQKWAFIYKLVDDPSQSSELLSEAKKLNDSQAPKSGGGGSGGGGSGGGGSLSGRSDNHGGGGSGGGGSGGGGSVDNKFNKEMWIAWEEIRDLPNLNGWQMTAFIASLLDDPSQSANLLAEAKKLNDAQAPKGGGGSLAEAKVLANRELDKYGVSDFYKRLINKAKTVEGVEALKLHILAALPEEEC

SEQ ID NO: 13 - protein, 6 aa, Staphylococcus carnosus
GILGVP

SEQ ID NO: 14 - protein, 8 aa, Staphylococcus carnosus
GPLGIAGQ

SEQ ID NO: 15 - protein, 10 aa, Staphylococcus carnosus
VHMPLGFLGP

SEQ ID NO: 16 - protein, 15 aa, Staphylococcus carnosus
SGGPGPAGMKGLPGS

SEQ ID NO: 17 - protein, 6 aa, Staphylococcus carnosus
PLGLAG

SEQ ID NO: 18 - protein, 6 aa, Staphylococcus carnosus
LALGPG

SEQ ID NO: 19 - protein, 8 aa, Staphylococcus carnosus
KRALGLPG

SEQ ID NO: 20 - protein, 5 aa, Staphylococcus carnosus
GGGRR

SEQ ID NO: 21 - protein, 8 aa, Staphylococcus carnosus
LSGRSDNH

SEQ ID NO: 22 - protein, 5 aa, Staphylococcus carnosus
PMAKK

SEQ ID NO: 23 - protein, 8 aa, Staphylococcus carnosus
RQARVVNG

SEQ ID NO: 24 - protein, 7 aa, Staphylococcus carnosus
HSSKLQL

SEQ ID NO: 25 - protein, 8 aa, Staphylococcus carnosus
RRSSYYSG

SEQ ID NO: 26 - protein, 57 aa, Staphylococcus carnosus
VDAKYAKEIRSATEIWWLPNLTADQKWAFIYKLVDDPSQSSELLSEAKKLNDSQAPK

SEQ ID NO: 27 - protein, 38 aa, Staphylococcus carnosus
SGGGGSGGGGSGGGGSENLYFQGGGGGSGGGGSGGGGS

SEQ ID NO: 28 - protein, 6 aa, Staphylococcus carnosus
VDLQAC

SEQ ID NO: 29 - protein, 201 aa, Staphylococcus carnosus
KLLDALAKAKADALKEFNKYGVSDYYKNLINNAKTVEGVKDLQAQVVESAKKARISEATDGLSDFLKSQTPAEDTVKSIELAEAKVLANRELDKYGVSDYHKNLINNAKTVEGVKDLQAQVVESAKKARISEATDGLSDFLKSQTPAEDTVKSIELAEAKVLANRELDKYGVSDYYKNLINNAKTVEGVKALIDEILAALP

SEQ ID NO: 30 - protein, 6 aa, Staphylococcus carnosus
HEHEHE

SEQ ID NO: 31 - protein, 39 aa, Staphylococcus carnosus
SGGGGSGGGGSGGGGSLSGRSDNHGGGGSGGGGSGGGGS

SEQ ID NO: 32 - protein, 5 aa, Staphylococcus carnosus
GGGGS

SEQ ID NO: 33 - protein, 4 aa, Staphylococcus carnosus
EEEC

SEQ ID NO: 34 - protein, 4 aa, Staphylococcus carnosus
GSSC

SEQ ID NO: 35 - protein, 6 aa, Staphylococcus carnosus
HHHHHH

SEQ ID NO: 36 - protein, 7 aa, Staphylococcus carnosus
ENLYFQG

SEQ ID NO: 37 - protein, 4 aa, Staphylococcus carnosus
GGGS

SEQ ID NO: 38 - protein, 8 aa, Staphylococcus carnosus
MSGRSANA

(19) United States
(12) Patent Application Publication
(10) Pub. No.: US 2025/0215093 A1
(43) Pub. Date: Jul. 3, 2025

(54) FUSION PROTEIN COMPRISING AN EGFR-BINDING DOMAIN AND A MASKING DOMAIN

(71) Applicant: ZYTOX THERAPEUTICS AB, Johanneshov (SE)

(72) Inventors: Stefan STÅHL, Stockholm (SE); Charles DAHLSSON LEITAO, Stockholm (SE); John LÖFBLOM, Tullinge (SE); Anna MESTRE BORRAS, Stockholm (SE)

(21) Appl. No.: 18/834,703

(22) PCT Filed: Feb. 6, 2023
(86) PCT No.: PCT/EP2023/052882

§ 371 Date: Jul. 31, 2024

(30) Foreign Application Priority Data
Feb. 4, 2022 (SE) 2250114-2

(51) Int. Cl. C07K 16/28; A61K 51/08; A61K 51/10

(52) U.S. Cl. CPC C07K 16/2863; A61K 51/088; A61K 51/103; C07K 2319/31; C07K 2319/50; C07K 2319/70

(57) ABSTRACT

There is provided a fusion protein comprising an EGFR-binding domain, a masking domain and a linker linking the masking domain to the EGFR-binding domain.

### TECHNICAL FIELD

The present disclosure relates to selective targeting of EGFR expressed on tumor cells.

### BACKGROUND

Overexpression of oncogenic receptors is common in cancer and presents an opportunity for designing tumor-selective drugs. However, in many cases, these receptors are also often abundantly expressed in healthy tissues thus limiting the potential of therapies in terms of safety and efficacy. The epidermal growth factor receptor (EGFR or ErbB-1) is a tyrosine kinase receptor involved in tumorigenesis for many cancers and is often due to overexpression. Therapeutic targeting of this receptor has been marked with challenges of systemic toxicity due to its abundant endogenous expression. Designing drugs that are more active in the tumor microenvironment and less active in circulation and healthy tissues would have the potential to significantly improve toxicity profiles and efficacies, allowing for more aggressive treatment options otherwise limited by systemic toxicity.

### SUMMARY

Affibody molecules are small (58 amino acid residues, 6.5 kDa) three-helical affinity proteins and promising alternatives to antibody-based drugs because of efficient tissue penetration, high stability, simple modularity of functional domains and ease of production in prokaryotic hosts. Novel binding specificities are generated from large combinatorial libraries by randomization of surface exposed residues on helices 1 and 2 of the affibody molecule (FIG. 1). Common selection platforms include phage and cell-surface display, with cell-surface display providing the possibility of using fluorescence-assisted cell sorting (FACS) to isolate binding populations and discriminate between closely related affinities. An affibody library with a size of 109 variants displayed on gram-positive Staphylococcus carnosus (Löfblom et al. Appl. Microbiol. Biotechnol. 2017,101 (23-24), 8293-8307) has previously been used to generate novel affibody binders. The library was synthesized using trinucleotide codons to avoid sequence bias and stop codons. This system takes advantage of the XM cell-wall anchoring sequence derived from staphylococcal protein A to display the affibody library. Cells displaying affibody variants are incubated with soluble fluorescently labelled target-of-interest and subsequently sorted using FACS based on fluorescent signal. Two albumin-binding domains are included in the displayed protein construct for normalization of cell-surface affibody expression using differentially labelled fluorescent albumin to provide a linear correlation between affinity and fluorescent signal. Magnetic-assisted cell sorting (MACS) can be used prior to FACS as a pre-enrichment step to reduce complexity of the library, which uses magnetic beads with immobilized target to capture cells displaying binding affibody variants.

The present disclosure is based on the development a conditionally activated affibody-based prodrug targeting EGFR. To achieve conditional activation, Staphylococcus carnosus cell-surface display was used to select for an anti-idiotypic affibody molecule masking the binding interface of a preexisting EGFR-targeting affibody molecule (ZEGFR:2377, see: Friedman et al., J. Mol. Biol. 2008, 376 (5), 1388-1402; Tolmachev et al. Eur. J. Nucl. Med. Mol. Imaging 2010, 37(3), 613-622 and WO 2007/065635). ZEGFR:2377 is hereinafter referred to as “ZEGFR”.

A masking domain (“ZB05”) showing high binding propensity in flow cytometry (FC) on the surface of S. carnosus has been isolated. The ZB05 affibody was produced as a soluble monomer and characterized in terms of binding to ZEGFR and thermostability. From kinetic evaluation using SPR, rapid association and dissociation rates were observed, which are favorable for the utility of a masking domain. Additionally, the protein demonstrated high thermostability (Tₘ 64.1° C.) and refolding capacity following heat denaturation, a common trait seen for monomeric affibody molecules.

The binding contribution of various residues of ZB05 was investigated by mutations. Results from the mutagenesis study revealed flexibility and interchangeability of residues in several randomized positions in terms of retained binding (see FIG. 10).

To further investigate the potential of ZB05 as a masking domain, a proof-of-concept pro-affibody (POC-PA) construct was designed in which ZB05 was fused to ZEGFR-ABP using a TEV-cleavable linker (ABP means “albumin-binding protein”). The capacity to mask EGFR-binding was initially analyzed using FC as a displayed protein on staphylococcal cells in the presence of fluorescently labelled soluble EGFR. Binding to EGFR could not be observed for cells displaying the intact protein but after treating the cells with TEV-protease the binding to EGFR was restored.

The construct was produced as a soluble molecule and evaluated for cleavage by TEV protease. After 1 hour incubation with TEV protease, the protein was completely digested showing distinct bands of correct size on an SDS-page gel. Binding to recombinant immobilized EGFR on Surface Plasmon resonance (SPR) was similarly masked by ZB05 for intact POC-PA and restored for cleaved POC-PA. A separate surface immobilized with human serum albumin was used to confirm equal injection amounts for intact and cleaved POC-PA.

Intact and cleaved POC-PA were tested for binding to endogenously expressed EGFR on H292 human mucoepidermoid pulmonary carcinoma cells and A431 human squamous carcinoma cells expressing moderate to high levels of EGFR respectively. Binding relative to cells alone could be observed not only for cleaved POC-PA, but also for intact POC-PA. Nonetheless, cleaved POC-PA increased the signal for both cell lines and was comparable to the construct POC-PA-DM where the ZB05 had been exchanged for a dummy masking domain without specificity for ZEGFR.

The capacity of an [¹¹¹In]In-labeled prodrug along with a variant thereof having a non-cleavable linker (“dummy-linker”) and a non-masked control to target EGFR-expressing matriptase-positive cancer cells in vivo without binding to EGFR-expressing hepatocytes was evaluated using a radioactive label. For labelling using ¹¹¹In, a maleimido derivative of DOTA-chelator was conjugated to C-termini of all constructs. This site-specific approach ensures that the radioactive label reflects the distribution of ZEGFR-ABD035-fusion. Additionally, the site-specific labelling provides a homogenously labelled protein, not a mixture of proteins with different numbers of conjugated chelators in different positions. All radiolabeled constructs had a high radiochemical purity and demonstrated excellent stability. The in vitro tests demonstrated that the binding of [¹¹¹In]In-labeled non-masked control to both cell lines is significantly reduced by saturation of receptors using both non-labelled ZEGFR-ABD035-fusion and cetuximab, which shows that the binding was specific. In vitro binding of [¹¹¹In]In-labeled prodrug and dummy-linker was much lower and predominantly unspecific. This suggests that the incorporation of anti-idiotypic masking domain efficiently prevents binding of these constructs to EGFR in vitro.

Initial in vivo evaluation of the [¹¹¹In]In-labeled prodrug, the dummy-linker and the non-masked control demonstrated that concentration of all these proteins in blood was much higher that the concentration of non-ABD035-fused ¹¹¹In-ZEGFR and the renal uptake was much lower. This phenomenon demonstrates that there is a binding of the ABD035 to the murine albumin in vivo. This prevents glomerular filtration of the constructs and their reabsorption in proximal tubuli. Accordingly, the bioavailability of the constructs is higher and potential renal toxicity is lower compared to targeted constructs based on non-ABD035-fused ZEGFR. The most important observation from this experiment concerns the hepatic uptake. The uptake of non-masked control in liver was high, 17.2±1 and 12.7±1.8% ID/g at 4 and 24 h after injection, respectively. This is expected because of a noticeable expression of EGFR on hepatocytes and high affinity of ZEGFR to murine EGFR. The prodrug and the dummy-linker had much lower hepatic uptake, which shows that the incorporation of the masking domain served its purpose.

Data from the experiment in mice bearing EGFR-expressing H292 xenografts confirmed extended residence time in circulation of all constructs and low hepatic uptake of the [¹¹¹In]In-labeled prodrug and the dummy-linker. Importantly, the uptake of the [¹¹¹In]In-Prodrug in H292 xenografts was significantly higher than in EGFR-negative Ramos xenografts. This was also confirmed in the imaging experiment. This demonstrates that uptake in H292 xenografts was EGFR-specific. Another interesting finding is that the tumor uptake was equally high for the prodrug and the dummy-linker.

To elucidate the role of matriptases in the tumor uptake of the [¹¹¹In]In-labeled prodrug, an additional experiment was performed. The tumor uptake was compared in mice bearing H292 and A431 xenografts simultaneously. The results of this experiment demonstrated that the uptake in H292 xenografts with high matriptase expression was two times higher than in A431 xenografts with low matriptase expression. At the same time, the EGFR expression per cell is three-fold higher in A431 cells than H292 cells. Taken together, these data suggests that matriptases play a role in the tumor uptake of the [¹¹¹In]In-labeled prodrug. At the same time, the uptake in A431 xenograft was higher than in EGFR-negative Ramos xenografts, which suggests that a matriptase-independent mechanism is also in play.

In conclusion, the data show that the masked EGFR binders have a significantly lower liver uptake than a non-masked version and, interestingly, that tumor uptake of the masked EGFR binders was on the same level as that of the non-masked version independent of a protease-cleavable linker. The masking domain thus improves the tumor to liver ratio significantly.

Accordingly, the following itemized listing of embodiments of the present disclosure is provided.

1. A fusion protein comprising an EGFR-binding domain, a masking domain and a linker linking the masking domain to the EGFR-binding domain, wherein:
the masking domain comprises the amino acid sequence

IX₁₀SX₁₂X₁₃X₁₄X₁₅X₁₆WWX₁₉X₂₀X₂₁X₂₂X₂₃X₂₄X₂₅X₂₆KX₂₈X₂₉X₃₀X₃₁YX₃₃
X₃₄V

wherein, independently of each other,
X₁₀ is R, K, M, N or Q;
X₁₂ is A or a substitution;
X₁₃ is E or absent;
X₁₄ is T or S;
X₁₅ is E or a substitution;
X₁₆ is I or a substitution;
X₁₉ is L, a substitution or absent;
X₂₀ is P, a substitution or absent;
X₂₁ is N, a substitution or absent;
X₂₂ is L, a substitution or absent;
X₂₃ is T or a substitution;
X₂₄ is A, F, I, K, L, M, T or Y;
X₂₅ is D, G, I or W;
X₂₆ is Q or a substitution;
X₂₈ is W, A, F, I, L, M, Q, R, S, T or V;
X₂₉ is A or a substitution;
X₃₀ is F or a substitution;
X₃₁ is I or L;
X₃₃ is K or a substitution; and
X₃₄ is L or a substitution,
provided that no more than six of X₁₂, X₁₅, X₁₆, X₁₉, X₂₀, X₂₁, X₂₂, X₂₃, X₂₆, X₂₉, X₃₀, X₃₃ and X₃₄ are substitutions and that no more than two of X₁₉-X₂₂ are absent; and
the EGFR-binding domain comprises the amino acid sequence

EX₂X₃X₄AX₆X₇EIX₁₀X₁₁LPNLNX₁₇X₁₈QX₂₀X₂₁AFIX₂₅SLX₂₈D,

wherein, independently of each other,
X₂ is M, F, V, L, I or S;
X₃ is W, D, E or L;
X₄ is I, V, G, S, M, L, A, T, N, D or W;
X₆ is W, V, L, I, M or S;
X₇ is D, E, N or K;
X₁₀ is R, G, H or K;
X₁₁ is D, N, E, Y or S;
X₁₇ is G, W or A;
X₁₈ is W, G or AA;
X₂₀ is M, L, F, A or E;
X₂₁ is T, D, N, A or Q;
X₂₅ is A, S, N, G or L; and
X₂₈ is L, W, V, F or A.

2. The fusion protein of item 1, wherein, in the masking domain, no more than four of X₁₂, X₁₅, X₁₆, X₁₉, X₂₀, X₂₁, X₂₂, X₂₃, X₂₆, X₂₉, X₃₀, X₃₃ and X₃₄ are substitutions and/or no more than one of X₁₉-X₂₂ is absent.

3. The fusion protein of item 2, wherein, in the masking domain, no more than two of X₁₂, X₁₅, X₁₆, X₁₉, X₂₀, X₂₁, X₂₂, X₂₃, X₂₆, X₂₉, X₃₀, X₃₃ and X₃₄ are substitutions and/or none of X₁₉-X₂₂ is absent.

4. The fusion protein of any one of the preceding items, wherein the masking domain comprises the amino acid sequence

X₅X₆X₇X₈IX₁₀SX₁₂X₁₃X₁₄X₁₅X₁₆WWX₁₉X₂₀X₂₁X₂₂X₂₃X₂₄X₂₅X₂₆KX₂₈X₂₉
X₃₀X₃₁YX₃₃X₃₄V

wherein, independently of each other,
X₅ is Y or a substitution;
X₆ is A or a substitution;
X₇ is K or a substitution; and
X₈ is E or a substitution,
provided that no more than two of X₅-X₈ are substitutions.

5. The fusion protein of item 4, wherein the masking domain comprises the amino acid sequence

X₁X₂X₃X₄X₅X₆X₇X₈IX₁₀SX₁₂X₁₃X₁₄X₁₅X₁₆WWX₁₉X₂₀X₂₁X₂₂X₂₃X₂₄X₂₅X₂₆K
X₂₈X₂₉X₃₀X₃₁YX₃₃X₃₄V

wherein, independently of each other,
X₁ is V or a substitution;
X₂ is D or a substitution;
X₃ is A or a substitution; and
X₄ is K or a substitution,
provided that no more than four of X₁-X₈ are substitutions.

6. The fusion protein of item 5, wherein, in the masking domain, no more than two of X₁-X₈ are substitutions.

7. The fusion protein of any one of the preceding items, wherein the masking domain comprises the amino acid sequence

IX₁₀SX₁₂X₁₃X₁₄X₁₅X₁₆WWX₁₉X₂₀X₂₁X₂₂X₂₃X₂₄X₂₅X₂₆KX₂₈X₂₉X₃₀X₃₁YX₃₃
X₃₄VX₃₆X₃₇X₃₈X₃₉X₄₀X₄₁X₄₂X₄₃X₄₄

wherein, independently of each other,
X₃₆ is D or a substitution;
X₃₇ is D or a substitution;
X₃₈ is P or a substitution;
X₃₉ is S or a substitution;
X₄₀ is Q or a substitution;
X₄₁ is S or a substitution;
X₄₂ is S or a substitution;
X₄₃ is E or a substitution;
X₄₄ is L or a substitution;
provided that no more than four of X₃₆-X₄₄, are substitutions.

8. The fusion protein of any one of the preceding items, wherein, in the masking domain, no more than two of X₃₆-X₄₄ are substitutions.

9. The fusion protein item 7 or 8, wherein the masking domain comprises the amino acid sequence

IX₁₀SX₁₂X₁₃X₁₄X₁₅X₁₆WWX₁₉X₂₀X₂₁X₂₂X₂₃X₂₄X₂₅X₂₆KX₂₈X₂₉X₃₀X₃₁YX₃₃
X₃₄VX₃₆X₃₇X₃₈X₃₉X₄₀X₄₁X₄₂X₄₃X₄₄X₄₅X₄₆X₄₇X₄₈X₄₉X₅₀X₅₁X₅₂X₅₃X₅₄

wherein, independently of each other,
X₄₅ is L or a substitution;
X₄₆ is S or a substitution;
X₄₇ is E or a substitution;
X₄₈ is A or a substitution;
X₄₉ is K or a substitution;
X₅₀ is K or a substitution;
X₅₁ is L or a substitution;
X₅₂ is N or a substitution;
X₅₃ is D or a substitution,
X₅₄ is S or a substitution;
provided that no more than five of X₄₅-X₅₄ are substitutions.

10. The fusion protein of item 9, wherein, in the masking domain, no more than seven of X₃₆-X₅₄ are substitutions.

11. The fusion protein of item 10, wherein, in the masking domain, no more than five of X₃₆-X₅₄ are substitutions.

12. The fusion protein of any one of items 9-11, wherein the masking domain comprises the amino acid sequence

IX₁₀SX₁₂X₁₃X₁₄X₁₅X₁₆WWX₁₉X₂₀X₂₁X₂₂X₂₃X₂₄X₂₅X₂₆KX₂₈X₂₉X₃₀X₃₁YX₃₃
X₃₄VX₃₆X₃₇X₃₈X₃₉X₄₀X₄₁X₄₂X₄₃X₄₄X₄₅X₄₆X₄₇X₄₈X₄₉X₅₀X₅₁X₅₂X₅₃X₅₄X₅₅
X₅₆X₅₇X₅₈

wherein, independently of each other,
X₅₅ is Q or a substitution,
X₅₆ is A or a substitution;
X₅₇ is P or a substitution;
X₅₈ is K or a substitution;
provided that no more than two of X₅₅-X₅₈ are substitutions.

13. The fusion protein of item 12, wherein, in the masking domain, no more than seven of X₃₆-X₅₈ are substitutions.

14. The fusion protein of item 13, wherein, in the masking domain, no more than five of X₃₆-X₅₈ are substitutions.

15. The fusion protein of any one of the preceding items, wherein, in the masking domain, X₁₀ is R.

16. The fusion protein of any one of the preceding items, wherein, in the masking domain, X₁₃ is absent.

17. The fusion protein of any one of the preceding items, wherein, in the masking domain, X₁₄ is T.

18. The fusion protein of any one of the preceding items, wherein, in the masking domain, X₂₄ is A.

19. The fusion protein of any one of the preceding items, wherein, in the masking domain, X₂₅ is D.

20. The fusion protein of any one of the preceding items, wherein, in the masking domain, X₂₈ is W.

21. The fusion protein of any one of the preceding items, wherein, in the masking domain, X₃₁ is I.

22. The fusion protein of any one of the preceding items, wherein the masking domain comprises the amino acid sequence

IRSX₁₂TX₁₅X₁₆WWX₁₉X₂₀X₂₁X₂₂X₂₃ADX₂₆KWX₂₉X₃₀IYX₃₃X₃₄V.

23. The fusion protein of any one of the preceding items, wherein the masking domain comprises the amino acid sequence IRSATEIWWLPNLTADQKWAFIYKLV (SE ID NO:1).

24. The fusion protein of any one of the preceding items, wherein, in the EGFR-binding domain, X₃ is W.

25. The fusion protein of any one of the preceding items, wherein, in the EGFR-binding domain, X₆ is V or W.

26. The fusion protein of any one of the preceding items, wherein, in the EGFR-binding domain, X₁₀ is R or G.

27. The fusion protein of any one of the preceding items, wherein, in the EGFR-binding domain, X₁₀ is W or G.

28. The fusion protein of any one of the preceding items, wherein, in the EGFR-binding domain, X₁₈ is W or G, preferably W.

29. The fusion protein of any one of the preceding items, wherein, in the EGFR-binding domain, X₂₁ is T or D, preferably T.

30. The fusion protein of any one of the preceding items, wherein the EGFR-binding domain comprises the amino acid sequence

EX₂WX₄AWX₇EIRXILPNLNGWQX₂₀TAFIX₂₅SLX₂₈D,

wherein, independently of each other,
X₂ is M, V, L or I;
X₄ is I, V, G, S, M, L, A, T, N or D;
X₇ is D, E, N or K;
X₁₁ is D, N, E, Y or S;
X₂₀ is M, L or F;
X₂₅ is A, S, or G; and
X₂₈ is L or V.

31. The fusion protein of any one of the preceding items, wherein, in the EGFR-binding domain, X₂ is M.

32. The fusion protein of any one of the preceding items, wherein, in the EGFR-binding domain, X₄ is I, V, G or S.

33. The fusion protein of any one of the preceding items, wherein, in the EGFR-binding domain, X₁₁ is D, N or E.

34. The fusion protein of any one of the preceding items, wherein, in the EGFR-binding domain, X₂₀ is M.

35. The fusion protein of any one of the preceding items, wherein, in the EGFR-binding domain, X₂₅ is A or S.

36. The fusion protein of any one of the preceding items, wherein, in the EGFR-binding domain, X₂₈ is L.

37. The fusion protein of any one of the preceding items, wherein, in the EGFR-binding domain, X₂ is M, X₂₀ is M and X₂₈ is L.

38. The fusion protein of any one of the preceding items, wherein the EGFR-binding domain comprises the amino acid sequence

(SEQ ID NO: 2)
EMWIAWEEIRDLPNLNGWQMTAFIASLLD.

39. The fusion protein of any one of the preceding items, wherein the EGFR-binding domain comprises the amino acid sequence

(SEQ ID NO: 3)
VDNKFNKEMWIAWEEIRDLPNLNGWQMTAFIASLLD.

40. The fusion protein of any one of the preceding items, wherein the EGFR-binding domain comprises the amino acid sequence

(SEQ ID NO: 4)
EMWIAWEEIRDLPNLNGWQMTAFIASLLDDPSQSANLLAEAKKLNDAQAP
K.

41. The fusion protein of any one of the preceding items, wherein the EGFR-binding domain comprises the amino acid sequence

(SEQ ID NO: 5)
VDNKFNKEMWIAWEEIRDLPNLNGWQMTAFIASLLDDPSQSANLLAEAKK
LNDAQAPK.

42. The fusion protein of any one of the preceding items further comprising a half-life-extending region, such as an Fc-binding region or an albumin-binding region (ABR).

43. The fusion protein of any one of the preceding items further comprising an albumin-binding region (ABR), which ABR comprises an amino acid sequence selected from
a) LAX₃AKX₆X₇AX₉X₁₀ ELDX₁₄YGVSDX₂₀ YKX₂₃LIX₂₆X₂₇AKT₃VEGVX₃₅ALX₃₈X₃₉X₄₀ ILX₄₃X₄₄X₄₅X₄₆, wherein, independently of each other,
   X₃ is selected from E, S, Q and C;
   X₆ is selected from E, S, V and C;
   X₇ is selected from A, L and S;
   X₉ is selected from L and N;
   X₁₀ is selected from A, S and R;
   X₁₄ is selected from A, S, C and K;
   X₂₀ is selected from Y and F;
   X₂₃ is selected from N, D and R;
   X₂₆ is selected from N, D and E;
   X₂₇ is selected from N and K;
   X₃₅ is selected from K and E;
   X₃₈ is selected from I and K;
   X₃₉ is selected from D, E and L;
   X₄₀ is selected from A, E and H;
   X₄₃ is selected from A and K;
   X₄₄ is selected from A, S and E;
   X₄₅ is L or absent; and
   X₄₆ is P or absent;
and b) an amino acid sequence which has at least 95% identity to the sequence defined in a).

44. The fusion protein of item 43, wherein the ABR comprises the amino acid sequence
LAX₃AKX₆X₇AX₉X₁₀ ELDX₁₄YGVSDX₂₀ YKX₂₃LIX₂₆X₂₇AKTVEGVX₃₅ALX₃₈X₃₉X₄₀ ILAALP, wherein, independently of each other,
   X₃ is selected from E and S;
   X₆ is selected from E and V;
   X₇ is selected from A and L;
   X₉ is selected from L and N;
   X₁₀ is selected from A and R;
   X₁₄ is selected from A, S, C and K, preferably from A, S and K;
   X₂₀ is selected from Y and F;
   X₂₃ is selected from N, D and R;
   X₂₆ is selected from N and D;
   X₂₇ is selected from N and K;
   X₃₅ is selected from K and E;
   X₃₈ is selected from I and K;
   X₃₉ is selected from D and L;
   X₄₀ is selected from A, E and H;
   X₄₅ is L or absent; and
   X₄₆ is P or absent.

45. The therapeutic conjugate according to item 43, wherein the ABR comprises an amino acid sequence selected from the group consisting of:

(SEQ ID NO: 6)
LAEAKVLANR ELDKYGVSDF YKRLINKAKT VEGVEALKLH
ILAALP;
(SEQ ID NO: 7)
LAEAKEAANA ELDSYGVSDF YKRLIDKAKT VEGVEALKDA
ILAALP;
(SEQ ID NO: 8)
GLAEAKEAAN AELDSYGVSD FYKRLIDKAK TVEGVEALKD
AILAALP;
(SEQ ID NO: 9)
LAEAKVLANR ELDKYGVSDY YKNLINNAKT VEGVKALIDE
ILAALP;
and
(SEQ ID NO: 10)
LAEAKVLALR ELDKYGVSDY YKDLIDKAKT VEGVKALIDE
ILAALP.

46. The fusion protein of any one of items 42-45, wherein the half-life-extending region is located on the C-terminal side of the EGFR-binding domain.

47. The fusion protein of any one of the preceding items, wherein the linker is a protease-cleavable linker.

48. The fusion protein of item 47, wherein the protease-cleavable linker comprises a sequence selected from the group consisting of GFLG (SEQ ID NO:11), Glutamic acid-Valine-Citrulline, GILGVP (SEQ ID NO:13), GPLGIAGQ (SEQ ID NO:14), VHMPLGFLGP (SEQ ID NO:15), SGGPGPAGMKGLPGS (SEQ ID NO: 16), PLGLAG (SEQ ID NO:17), LALGPG (SEQ ID NO:18), KRALGLPG (SEQ ID NO: 19), GGGRR (SEQ ID NO:20), LSGRSDNH (SEQ ID NO:21), PMAKK (SEQ ID NO:22), RQARVVNG (SEQ ID NO:23), MSGRSANA (SEQ ID NO:38), HSSKLQL (SEQ ID NO:24) and RRSSYYSG (SEQ ID NO:25).

49. The fusion protein of any one of the preceding items, wherein the length of the linker is at least 12 amino acid residues, such as 12-60 amino acid residues, such as 20-50 amino acid residues.

50. The fusion protein of any one of the preceding items, wherein the linker comprises no cysteine (C) residue.

51. The fusion protein of any one of the preceding items, wherein the masking domain comprises no cysteine (C) residue.

52. The fusion protein of any one of the preceding items, wherein the EGFR-binding domain comprises no cysteine (C) residue.

53. The fusion protein of any one of the preceding items, wherein masking domain is located on the N-terminal side of the EGFR-binding domain.

54. A therapeutic conjugate comprising the fusion protein of any one of the preceding items and a cytotoxic agent, such as a cytotoxic molecule, peptide, protein or radionuclide.

55. The therapeutic conjugate according to item 54, wherein the cytotoxic agent is a cytotoxic radionuclide selected from the group consisting of ¹⁷⁷Lu, ⁹⁰Y, ¹⁸⁸Re; ¹⁸⁶Re; ¹⁶⁶Ho, ¹⁵³Sm, ⁶⁷Cu, ⁶⁴Cu, ¹⁴⁹Tb, ¹⁶¹Tb, ⁴⁷Sc; ²²⁵Ac; ²¹²Pb; ²¹³Bi, ²¹²Bi, ²²⁷Th, ²²³Ra; ^{58m}Co, ¹³¹I, ⁷⁶As, ⁷⁷As and ²¹¹At.

56. The therapeutic conjugate according to item 54, wherein the cytotoxic agent is a cytotoxic molecule selected from the group consisting of Doxorubicin (DOX), Duocarmycins (DUO), Docetaxel (DTX), Monomethyl auristatin E (MMAE), Monomethyl auristatin F (MMAF), Paclitaxel (FTX), Mertansine (DM1), emtansine (DM1), Ravtansine (DM4), Soravtansine (DM4), Pyrrolobenzodiazepine (PBD) and Calicheamicin.

57. The therapeutic conjugate according to item 54, wherein the cytotoxic agent is a protein-based toxin selected from the group consisting of Pseudomonas exotoxin (PE), Diphtheria toxin (DT), Ricin toxin A-chain (RTA) and deBouganin.

58. A therapeutic conjugate according to any one of items 54-57 for use in a therapeutic method of treatment.

59. The therapeutic conjugate for use according to item 58, wherein the therapeutic method of treatment is a method of treatment of a subject suffering from a cancer, such as a cancer overexpressing EGFR.

60. The therapeutic conjugate for use according to item 59, wherein the cancer is selected from the group consisting of lung cancer, preferably non-small cell lung cancer, prostate cancer, breast cancer, colon and rectum cancer, head and neck cancer, esophagogastric cancer, liver cancer, glioblastoma, cervix cancer, ovary cancer, bladder cancer, kidney cancer and pancreatic cancer.

### BRIEF DESCRIPTION OF THE FIGURES

FIG. 1 is a schematic overview of an affibody molecule. The affibody molecule shows the 14 surface-exposed amino acid positions that are randomized in the staphylococcal display library.

FIG. 2 is a schematic overview of a proof-of concept pro-affibody (POC-PA) construct. The POC-PA construct comprises an anti-idiotypic masking domain with specificity for an EGFR-binding affibody molecule, a TEV protease-cleavable linker, an EGFR-binding affibody domain and an albumin-binding protein.

FIG. 3 shows a characterization of the ZB05 affibody masking domain: (A) Melting curve (VTM) (left) and circular dichroism spectroscopy showing refolding capacity after heat denaturation of ZB05 (right); (B) SPR sensorgram showing binding interactions of B05 with immobilized ZEGFR (left) and negative control Human serum albumin (HSA) (right).

FIG. 4 shows a characterization of POC-PA produced as a soluble monomer: (A) SDS-page showing purity after HSA-affnity purification, the size of TEV-protease and the size of cleaved products following treatment with TEV-protease; and (B) analysis of protease-dependent binding of POC-PA to immobilized human EGFR (top) and unaffected binding to immobilized HSA (bottom) using SPR.

FIG. 5 shows a flow cytometric analysis of EGFR-binding on H292 and A431 cells for intact POC-PA and POC-PA pre-cleaved with TEV protease. A control construct (POC-PA-DM) with a dummy masking domain (ZE01) was included for comparison.

FIG. 6 shows the binding specificity of [111In]In-labeled prodrug, dummy-linker and non-masked control in (A) A431 cells and (B) H292 cells. Cells were incubated at room temperature with radiolabeled compounds with or without pre-saturation with a 100-fold molar excess of the non-masked control without the label. The Y-axis corresponds to the measured total activity of the cells as a percentage of the total added activity to each well. Asterisk (*) correspond to significant differences (p<0.05, t-test).

FIG. 7 shows a comparison of [111In]In-labeled non-masked control binding to H292 and A431 cells in vitro. Cells were incubated at room temperature with radiolabeled compounds (1.64×106 CPM) with or without pre-saturation with a 100-fold molar excess of the same compound lacking radiolabel or anti-EGFR antibody cetuximab, blocked or non-blocked, respectively. An additional set of dishes was used to count number of cells pre dish at the time of experiment.

FIG. 8 shows a comparison (t-test) of [111In]In-prodrug uptake in H292 (EGFR-positive) xenograft and Ramos (EGFR-negative) xenograft at (A) 4 h and (B) 48 h p.i.; (C) uptake in H292 (EGFR-positive, matriptase high level) and A431 (EGFR-positive, matriptase low level) in the same animals xenograft at 48 h p.i. Symbols (x) show the uptake in H292 xenografts; symbols (+) show the uptake in Ramos xenografts; symbols (●) show the uptake in A431 xenografts.

FIG. 9 shows Micro-Single-Photon Emission Computed Tomography/Computed Tomography (microSPECT/CT) imaging using [¹¹¹In]In-labeled non-masked control (left) and prodrug (right) in Balb/c nu/nu mice bearing EGFR-positive H292 xenograft at (A) 4 h and (C) 48 h p.i.. [¹¹¹In]In-labeled prodrug in Balb/c nu/nu mice bearing EGFR negative Ramos xenograft at (B) 4 h and (D) 48 h p.i.. The T arrows point at the tumor. The L arrows point at the liver.

FIG. 10 is a table showing allowed substitutions in each randomized position of the ZB05 scaffold that retain binding to ZEGFR. Allowed substitutions that retained binding was determined from a twofold enrichment in the binding population compared to the naïve library with at least a 50% depletion for the corresponding variant in the non-binding population. Randomized positions of the ZB05 sequence are marked with arrows.

FIG. 11 shows representative FACS sorting of the ZB05 mutagenesis library containing a total of 253 different variants (left) and flow cytometric analysis of the binding population (right).

FIG. 12 shows the full amino acid sequence of the proof-of-concept pro-affibody (POC-PA) prepared and tested in Example 1. In the masking domain ZB05, a subsequence (SEQ ID NO: 1) of particular relevance for binding to ZEGFR is highlighted in grey. In the TEV-cleavable linker, the subsequence recognized by TEV is highlighted in grey. In ZEGFR (i.e. the EGFR-binding domain), a subsequence (SEQ ID NO:2) of particular relevance for binding to EGFR is highlighted in grey.

FIG. 13 shows the full amino acid sequence of the (proof-of-concept) prodrug used in Example 2. In the masking domain ZB05, a subsequence (SEQ ID NO: 1) of particular relevance for binding to ZEGFR is highlighted in grey. In the matriptase-cleavable linker, the subsequence (SEQ ID NO: 21) recognized by matriptase is highlighted in grey. In ZEGFR (i.e. the EGFR-binding domain), a subsequence (SEQ ID NO:2) of particular relevance for binding to EGFR is highlighted in grey.

FIG. 14 shows SDS-page gels after cleaving of the PA described in Example 3 below.

### DETAILED DESCRIPTION

As a first aspect of the present disclosure, there is provided a fusion protein comprising an EGFR-binding domain, a masking domain and a linker linking the masking domain to the EGFR-binding domain.

The masking domain binds to the EGFR-binding domain and thereby restricts binding to EGFR under certain conditions.

The masking domain comprises the amino acid sequence

IX₁₀SX₁₂X₁₃X₁₄X₁₅X₁₆WWX₁₉X₂₀X₂₁X₂₂X₂₃X₂₄X₂₅X₂₆KX₂₈X₂₉X₃₀X₃₁Y
X₃₃X₃₄V

wherein, independently of each other,
X₁₀ is R, K, M, N or Q;
X₁₂ is A or a substitution;
X₁₃ is E or absent;
X₁₄ is T or S;
X₁₅ is E or a substitution;
X₁₆ is I or a substitution;
X₁₉ is L, a substitution or absent;
X₂₀ is P, a substitution or absent;
X₂₁ is N, a substitution or absent;
X₂₂ is L, a substitution or absent;
X₂₃ is T or a substitution;
X₂₄ is A, F, I, K, L, M, T or Y;
X₂₅ is D, G, I or W;
X₂₆ is Q or a substitution;
X₂₈ is W, A, F, I, L, M, Q, R, S, Tor V;
X₂₉ is A or a substitution;
X₃₀ is F or a substitution;
X₃₁ is I or L;
X₃₃ is K or a substitution; and
X₃₄ is L or a substitution.

The amino acid residues of positions X₁₂, X₁₅, X₁₆, X₁₉, X₂₀, X₂₁, X₂₂, X₂₃, X₂₆, X₂₉, X₃₀, X₃₃ and X₃₄ are not believed to form part of the binding site actively interacting with the EGFR-binding domain. Hence substitutions and in some cases (X₁₉-X₂₂) even deletions (as compared to ZB05) are allowed in these positions. However, the three-dimensional structure is believed to be tweaked to such a degree that the binding capacity is lost if all or most of them are substituted or (in case of X₁₉-X₂₂) deleted. Hence no more than six of X₁₂, X₁₅, X₁₆, X₁₉, X₂₀, X₂₁, X₂₂, X₂₃, X₂₆, X₂₉, X₃₀, X₃₃ and X₃₄ can be substitutions and no more than two of X₁₉-X₂₂can be absent in the fusion protein of the first aspect.

In one embodiment of the masking domain, no more than four of X₁₂, X₁₅, X₁₆, X₁₉, X₂₀, X₂₁, X₂₂, X₂₃, X₂₆, X₂₉, X₃₀, X₃₃ and X₃₄ are substitutions. Preferably, no more than two of X₁₂, X₁₅, X₁₆, X₁₉, X₂₀, X₂₁, X₂₂, X₂₃, X₂₆, X₂₉, X₃₀, X₃₃ and X₃₄ are substitutions.

In an alternative or complementary embodiment, no more than one of X₁₉-X₂₂ is absent. Preferably, none of X₁₉-X₂₂ is absent.

In one embodiment, the masking domain comprises further amino acid residues (X₅-X₈) at the N-terminus. Hence the masking domain may comprise the amino acid sequence

X₅X₆X₇X₈IX₁₀SX₁₂X₁₃X₁₄X₁₅X₁₆WWX₁₉X₂₀X₂₁X₂₂X₂₃X₂₄X₂₅X₂₆KX₂₈X₂₉
X₃₀X₃₁YX₃₃X₃₄V

wherein, independently of each other,
X₅ is Y or a substitution;
X₆ is A or a substitution;
X₇ is K or a substitution; and
X₈ is E or a substitution,
provided that no more than two of X₅-X₈ are substitutions.

In one embodiment, the masking domain comprises still further amino acid residues (X₁-X₄) at the N-terminus. Hence the masking domain may comprise the amino acid sequence

X₁X₂X₃X₄X₅X₆X₇X₈IX₁₀SX₁₂X₁₃X₁₄X₁₅X₁₆WWX₁₉X₂₀X₂₁X₂₂X₂₃X₂₄X₂₅X₂₆
KX₂₈X₂₉X₃₀X₃₁YX₃₃X₃₄V

wherein, independently of each other,
X₁ is V or a substitution;
X₂ is D or a substitution;
X₃ is A or a substitution; and
X₄ is K or a substitution,
provided that no more than four of, such as no more than two of, X₁-X₈ are substitutions.

In one embodiment, the masking domain comprises further amino acid residues (X₃₆-X₄₄) at the C-terminus. Hence the masking domain may comprise the amino acid sequence

IX₁₀SX₁₂X₁₃X₁₄X₁₅X₁₆WWX₁₉X₂₀X₂₁X₂₂X₂₃X₂₄X₂₅X₂₆KX₂₈X₂₉X₃₀X₃₁Y
X₃₃X₃₄VX₃₆X₃₇X₃₈X₃₉X₄₀X₄₁X₄₂X₄₃X₄₄

wherein, independently of each other,
X₃₆ is D or a substitution;
X₃₇ is D or a substitution;
X₃₈ is P or a substitution;
X₃₉ is S or a substitution;
X₄₀ is Q or a substitution;
X₄₁ is S or a substitution;
X₄₂ is S or a substitution;
X₄₃ is E or a substitution;
X₄₄ is L or a substitution;
provided that no more than four of, such as no more than two of, X₃₆-X₄₄ are substitutions.

In one embodiment, the masking domain comprises still further amino acid residues (X₄₅-X₅₄) at the C-terminus. Hence the masking domain may comprise the amino acid sequence

IX₁₀SX₁₂X₁₃X₁₄X₁₅X₁₆WWX₁₉X₂₀X₂₁X₂₂X₂₃X₂₄X₂₅X₂₆KX₂₈X₂₉X₃₀X₃₁Y
X₃₃X₃₄VX₃₆X₃₇X₃₈X₃₉X₄₀X₄₁X₄₂X₄₃X₄₄X₄₅X₄₆X₄₇X₄₈X₄₉X₅₀X₅₁X₅₂X₅₃X₅₄

wherein, independently of each other,
X₄₅ is L or a substitution;
X₄₆ is S or a substitution;
X₄₇ is E or a substitution;
X₄₈ is A or a substitution;
X₄₉ is K or a substitution;
X₅₀ is K or a substitution;
X₅₁ is L or a substitution;
X₅₂ is N or a substitution;
X₅₃ is D or a substitution,
X₅₄ is S or a substitution;
provided that no more than five of, such as no more than three of, X₄₅-X₅₄ are substitutions.

In one embodiment of the masking domain, no more than seven of, such as no more than five of, X₃₆-X₅₄ are substitutions.

The masking domain may be further extended another four (X₅₅-X₅₈) amino acid residues at the C-terminus. Hence the masking domain may comprise the amino acid sequence

IX₁₀SX₁₂X₁₃X₁₄X₁₅X₁₆WWX₁₉X₂₀X₂₁X₂₂X₂₃X₂₄X₂₅X₂₆KX₂₈X₂₉X₃₀X₃₁Y
X₃₃X₃₄VX₃₆X₃₇X₃₈X₃₉X₄₀X₄₁X₄₂X₄₃X₄₄X₄₅X₄₆X₄₇X₄₈X₄₉X₅₀X₅₁X₅₂X₅₃X₅₄
X₅₅X₅₆X₅₇X₅₈

wherein, independently of each other,
X₅₅ is Q or a substitution,
X₅₆ is A or a substitution;
X₅₇ is P or a substitution;
X₅₈ is K or a substitution;
provided that no more than two of X₅₅-X₅₈ are substitutions.

In one embodiment of the masking domain, no more than seven of, such as no more than five of, X₃₆-X₅₈ are substitutions.

In a preferred embodiment of the masking domain:
X₁₀ is R;
X₁₃ is absent;
X₁₄ is T;
X₂₄ is A;
X₂₅ is D;
X₂₈ is W; and/or
X₃₁ is I.

Accordingly, the masking domain may comprise the amino acid sequence

IRSX₁₂TX₁₅X₁₆WWX₁₉X₂₀X₂₁X₂₂X₂₃ADX₂₆KWX₂₉X₃₀IYX₃₃X₃₄V.

In one embodiment, the masking domain comprises no cysteine (C) residue.

In one embodiment, the masking domain, just like ZB05, comprises the amino acid sequence IRSATEIWWLPNLTADQKWAFIYKLV (SEQ ID NO:1).

In one embodiment, the masking domain comprises the amino acid sequence VDAKYAKEIRSATEIWWLPNLTADQKWAFIYKLVDDPSQSSELLSEAKKLNDSQAPK (SEQ ID NO:26), which is the sequence of ZB05.

The EGFR-binding domain of the fusion protein of the first aspect comprises the amino acid sequence

EX₂X₃X₄AX₆X₇EIX₁₀X₁₁LPNLNX₁₇X₁₈QX₂₀X₂₁AFIX₂₅SLX₂₈D,

wherein, independently of each other,
X₂ is M, F, V, L, I or S;
X₃ is W, D, E or L;
X₄ is I, V, G, S, M, L, A, T, N, D or W;
X₆ is W, V, L, I, M or S;
X₇ is D, E, N or K;
X₁₀ is R, G, H or K;
X₁₁ is D, N, E, Y or S;
X₁₇ is G, W or A;
X₁₈ is W, G or A;
X₂₀ is M, L, F, A or E;
X₂₁ is T, D, N, A or Q;
X₂₅ is A, S, N, G or L; and
X₂₈ is L, W, V, F or A.

The rationale behind this amino acid sequence (and the embodiments thereof presented below) is explained in WO 2007/065635, which is incorporated herein by reference.

In a preferred embodiment of the EGFR-binding domain:
X₃ is W;
X₆ is V or W;
X₁₀ is R or G;
X₁₇ is W or G;
X₁₈ is W or G, preferably W; and/or
X₂₁ is T or D, preferably T.

In one embodiment, the EGFR-binding domain comprises the amino acid sequence EX₂WX₄AWX₇EIRX₁₁LPNLNGWQX₂₀TAFIX₂₅SLX₂₈D,
wherein, independently of each other,
X₂ is M, V, L or I, preferably M;
X₄ is I, V, G, S, M, L, A, T, N or D, preferably I, V, G or S;
X₇ is D, E, N or K;
X₁₁ is D, N, E, Y or S, preferably D, N or E;
X₂₀ is M, L or F, preferably M;
X₂₅ is A, S, or G, preferably A or S; and
X₂₈ is L or V, preferably L.

In a preferred embodiment of the EGFR-binding domain, X₂ is M, X₂₀ is M and X₂₈ is L.

In a particularly preferred embodiment, the EGFR-binding domain comprises an amino acid sequence selected from:
(i) EMWIAWEEIRDLPNLNGWQMTAFIASLLD (SEQ ID NO:2); and
(ii) an amino acid sequence which has at least 90% identify, such as at least 95% identity, to the sequence defined in (i).

Optionally there are additional N-terminal amino acid residues such that the EGFR-binding domain comprises an amino acid sequence selected from:
(iii) VDNKFNKEMWIAWEEIRDLPNLNGWQMTAFIASLLD (SEQ ID NO:3); and
(iv) an amino acid sequence which has at least 90% identify, such as at least 95% identity, to the sequence defined in (iii).

Optionally there are additional C-terminal amino acid residues such that the EGFR-binding domain comprises an amino acid sequence selected from:
(v) EMWIAWEEIRDLPNLNGWQMTAFIASLLDDPSQSANLLAEAKKLNDAQAPK (SEQ ID NO:4); and
(vi) an amino acid sequence which has at least 90% identify, such as at least 95% identity, to the sequence defined in (v).

In one embodiment, there are additional amino acid residues at the N-terminus and the C-terminus such that the EGFR-binding domain comprises an amino acid sequence selected from:
(vii) VDNKFNKEMWIAWEEIRDLPNLNGWQMTAFIASLLDDPSQSANLLAFAKKLN DAQAPK (SEQ ID NO:5); and
(viii) an amino acid sequence which has at least 90% identify, such as at least 95% identity, to the sequence defined in (vii).

Sequence (vii) is the sequence of the EGFR-binding affibody used in the Examples section below (also referred to as ZEGFR).

In one embodiment, the EGFR-binding domain comprises no cysteine (C) residue.

In one embodiment of the fusion protein of the first aspect, the masking domain is located on the N-terminal side of the EGFR-binding domain. In this embodiment, the linker is thus linking the C-terminus of the masking domain to the N-terminus of the EGFR-binding domain.

In one embodiment of the fusion protein of the first aspect, the linker is a protease-cleavable linker. Preferably, such a linker is cleavable by one or more of the following proteases: Cathepsin B; MMP-2; MMP-9; MMP-7; Urokinase-type plasminogen activator; Matriptase; and Prostate-specific antigen (PSA). These proteases are found in tumor microenvironments.

The above-mentioned proteases recognize the cleaving sites listed below

### Cathepsin B

GFLG (SEQ ID NO:11; see ref [1]); and
Glutamic acid-Valine-Citrulline (see ref [2]).

### MMP-2/-9

GILGVP (SEQ ID NO:13; see ref [1]);
GPLGIAGQ (SEQ ID NO:14; see ref [1]);
VHMPLGFLGP (SEQ ID NO:15; see ref [3]);
SGGPGPAGMKGLPGS (SEQ ID NO:16; see ref [4]);
PLGLAG (SEQ ID NO:17; see ref [5]); and
LALGPG (SEQ ID NO:18; see ref [5]).

### MMP-7:

KRALGLPG (SEQ ID NO:19; see ref [1])

### Urokinase-Type Plasminogen Activator

GGGRR (SEQ ID NO:20; see ref [1]); and
LSGRSDNH (SEQ ID NO:21; see ref [6]).

### Matriptase

LSGRSDNH (SEQ ID NO:21; see refs [3] and [8]);
PMAKK (SEQ ID NO:22; see ref [3]);
RQARVVNG (SEQ ID NO:23; see ref [3]); and
MSGRSANA (SEQ ID NO:38; see ref [7]).

### Prostate-Specific Antigen (PSA)

HSSKLQL (SEQ ID NO:24; see ref [1]); and
RRSSYYSG (SEQ ID NO:25; see ref [1]).

Consequently, an embodiment of the protease-cleavable linker comprises at least one of these cleaving site sequences listed above.

### References (Proteases and Cleaving Sites)

[1] Choi, K. Y., Swierczewska, M., Lee, S. & Chen, X. Protease-activated drug development. Theranostics 2, 156-179 (2012).
[2] Anami, Y. et al. Glutamic acid-valine-citrulline linkers ensure stability and efficacy of antibody-drug conjugates in mice. Nat. Commun. 9, (2018).
[3] Geiger, M. et al. Protease-activation using anti-idiotypic masks enables tumor specificity of a folate receptor 1-T cell bispecific antibody. Nat. Commun. 11, 1-14 (2020).
[4] Panchal, A. et al. COBRA: a highly potent conditionally active T cell engager engineered for the treatment of solid tumors. MAbs 12, (2020).
[5] Trang, V. H. et al. A coiled-coil masking domain for selective activation of therapeutic antibodies. Nat. Biotechnol. 37, 761-765 (2019).
[6] Vasiljeva, O., Menendez, E., Nguyen, M., Craik, C. S. & Michael Kavanaugh, W. Monitoring protease activity in biological tissues using antibody prodrugs as sensing probes. Sci. Rep. 10, 5894 (2020).
[7] WO 2022/016270 A1.
[8] US2017/0204139A1.

To allow the masking sequence and the EGFR-binding sequence to bind to each other, the length of the linker is typically at least 12 amino acid residues, such as at least 20 amino acid residues. The maximum length may for example be 50 or 60 amino acid residues.

In one embodiment, the linker comprises no cysteine (C) residue.

The fusion protein of the first aspect may further comprise a half-life-extending region, such as an Fc-binding region or an albumin-binding region (ABR). The half-life-extending region may for example be located on the C-terminal side of the EGFR-binding domain.

Alternatively, a half-life-extending group is not part by the fusion protein, but connected to the fusion protein in another way. In this alternative embodiment, the fusion protein and the half-life-extending group together forms a construct that may comprise further parts or groups.

Various half-life-extending strategies for proteins are described in a review article by Kontermann (EXPERT OPINION ON BIOLOGICAL THERAPY, 2016 VOL. 16, NO. 7, 903-915).

In one embodiment, the fusion protein of the first aspect comprises an ABR comprising an amino acid sequence selected from
a) LAX₃AKX₆X₇AX₉X₁₀ ELDX₁₄YGVSDX₂₀ YKX₂₃LIX₂₆X₂₇AKT₃₅VEGVX₃₈ALX₃₈X₃₉X₄₀ ILX₄₃X₄₄X₄₅X₄₆, wherein, independently of each other,
   X₃ is selected from E, S, Q and C;
   X₆ is selected from E, S, V and C;
   X₇ is selected from A, L and S;
   X₉ is selected from L and N;
   X₁₀ is selected from A, S and R;
   X₁₄ is selected from A, S, C and K;
   X₂₀ is selected from Y and F;
   X₂₃ is selected from N, D and R;
   X₂₆ is selected from N, D and E;
   X₂₇ is selected from N and K;
   X₃₅ is selected from K and E;
   X₃₈ is selected from I and K;
   X₃₉ is selected from D, E and L;
   X₄₀ is selected from A, E and H;
   X₄₃ is selected from A and K;
   X₄₄ is selected from A, S and E;
   X₄₅ is L or absent; and
   X₄₆ is P or absent;
and b) an amino acid sequence which has at least 95% identity to the sequence defined in a).

The rationale behind this ABR sequence (and the embodiments thereof described below) is explained in WO 2021/180727.

The ABR preferably comprises the amino acid sequence LAX₃AKX₆X₇AX₉X₁₀ ELDX₁₄YGVSDX₂₀ YKX₂₃LIX₂₆X₂₇AKTVEGVX₃₅ALX₃₈X₃₉X₄₀ ILAALP, wherein, independently of each other,
X₃ is selected from E and S;
X₆ is selected from E and V;
X₇ is selected from A and L;
X₉ is selected from L and N;
X₁₀ is selected from A and R;
X₁₄ is selected from A, S, C and K, preferably from A, S and K;
X₂₀ is selected from Y and F;
X₂₃ is selected from N, D and R;
X₂₆ is selected from N and D;
X₂₇ is selected from N and K;
X₃₅ is selected from K and E;
X₃₈ is selected from I and K;
X₃₉ is selected from D and L;
X₄₀ is selected from A, E and H;
X₄₅ is L or absent; and
X₄₆ is P or absent.

One embodiment of the ABR comprises no cysteine (C) residue.

In one embodiment, the ABR comprises an amino acid sequence selected from the group consisting of:

(SEQ ID NO: 6, ABD035)
LAEAKVLANR ELDKYGVSDF YKRLINKAKT VEGVEALKLH
ILAALP;
(SEQ ID NO: 7)
LAEAKEAANA ELDSYGVSDF YKRLIDKAKT VEGVEALKDA
ILAALP;
(SEQ ID NO: 8, PEP07914 in WO2012004384)
GLAEAKEAAN AELDSYGVSD FYKRLIDKAK TVEGVEALKD
AILAALP;
(SEQ ID NO: 9, ABDwt)
LAEAKVLANR ELDKYGVSDY YKNLINNAKT VEGVKALIDE
ILAALP;
and
(SEQ ID NO: 10)
LAEAKVLALR ELDKYGVSDY YKDLIDKAKT VEGVKALIDE
ILAALP.

As a second aspect of the present disclosure, there is provided a therapeutic conjugate comprising the fusion protein of the first aspect and a cytotoxic agent, such as a cytotoxic molecule, peptide, protein or radionuclide. In case of a cytotoxic peptide or protein, the whole therapeutic conjugate may be a fusion protein.

The cytotoxic molecule may for example be Doxorubicin (DOX), Duocarmycins (DUO), Docetaxel (DTX), Monomethyl auristatin E (MMAE), Monomethyl auristatin F (MMAF), Paclitaxel (FTX), Mertansine (DM1), emtansine (DM1), Ravtansine (DM4), Soravtansine (DM4), Pyrrolobenzodiazepine (PBD) or Calicheamicin.

In one embodiment, the cytotoxic molecule is DM1, MMAE, MMAF or DM4 (see Tarcsa et al. Drug Discovery Today: Technologies; Volume 37, December 2020, Pages 13-22 and the review by Khongorzul et al. Mol Cancer Res; 18(1) January 2020).

Examples of cytotoxic proteins are Pseudomonas exotoxin (PE) and engineered variants thereof, including PE38, diphtheria toxin (DT) and deBouganin (see Antignani et al, Biomolecules; 2020 Sep. 17; 10(9):1331).

Other examples of cytotoxic proteins are targeting domains against immunomodulatory targets such as CD3, CD47, PD-1, PD-Li, CTLA-4, 4-1BB and OX40 (see the review by Blanco et al., Clin Cancer Res 2021 Oct. 15; 27(20):5457-5464).

The cytotoxic radionuclide may for example be selected from the group consisting of ¹⁷⁷Lu, ⁹⁰Y, ¹⁸⁸Re; ¹⁸⁶Re; ¹⁶⁶Ho, ¹⁵³Sm, ⁶⁷Cu, ⁶⁴Cu, ¹⁴⁹b, ¹⁶¹Tb, ⁴⁷Sc; ²⁵Ac; ²¹²Pb; ²¹³Bi, ²¹²Bi, ²²⁷Th, ²²³Ra; ^{58m}Co, ¹³¹I, ⁷⁶As, ⁷⁷As and ²¹¹At.

Preferred cytotoxic radionuclides are ¹⁷⁷Lu ⁹⁰Y and ¹⁸⁸Re (see the review by Rondon et al., Cancers (Basel); 2021 Nov. 7; 13(21):5570).

¹⁷⁷Lu, ⁹⁰Y, ¹⁸⁸Re; ¹⁸⁶Re; ¹⁶⁶Ho, ¹⁵³Sm, ⁶⁷Cu, ⁶⁴Cu, ¹⁴⁹⁷b, ¹⁶¹Tb, ⁴⁷Sc; ²²⁵Ac; ²¹²Pb; ²¹³Bi, ²¹²Bi, ²²⁷Th, ²²³Ra and ^{58m}Co are radiometals that may be bound to the fusion protein by means of chelator-based conjugation. The chelator is preferably covalently bound to a cysteine residue of the fusion protein, optionally via a thiol-reactive linker. Binding to an amine of an amino acid residue of the fusion protein is also possible, but generally less preferred.

The chelator may be selected from the group consisting of DOTA and its derivatives (e.g. the maleimido-derivative of DOTA), cross-bridged macrocyclic chelators and sterically-restricted acyclic chelators.

Particularly suitable chelators for ¹⁷⁷Lu, ⁹⁰Y, ¹⁶⁶Ho, ¹⁵³Sm, ¹⁴⁹Tb, ¹⁶¹Tb, ⁴⁷Sc; ²²⁵Ac; ²¹²Pb; ²¹³Bi, ²¹²Bi, ²²⁷Th and ^{58m}Co are DOTA and its derivative DOTAGA.

For ⁶⁷Cu and ⁶⁴Cu a cross-bridged chelator, such as CB-TE2A, is a better option.

For ¹⁸⁸Re and ¹⁸⁶Re a chelator based on a cysteine- or mercaptoacetyl-containing peptide is preferred.

¹³¹I, ⁷⁶As, ⁷⁷As and ²¹¹At are non-metal radionuclides that can be bound to the fusion protein by means of covalent conjugation.

The radioiodination may be achieved using ((4-hydroxyphenyl)ethyl) maleimide (HPEM), which can be bound to a cysteine (C) residue of the fusion protein. ⁷⁶As and ⁷⁷As (and ⁷⁴As) in As (III) form may be coupled directly to a (freshly) reduced thiol group of a cysteine (C) residue of the fusion protein.

For coupling of ²¹¹At, N-[4-(tri-n-butylstannyl) phenethyl]-maleimide can be used as a linker. In such case, ²¹¹At is first coupled to the linker by astatodestannylation forming 4-astato-phenethyl-maleimide (AtPEM), which in turn can be coupled to a cysteine (C) residue of the fusion protein.

Consequently, the cytotoxic radionuclide is preferably linked to the fusion protein via a cysteine (C) residue of the fusion protein. To avoid cross/side reactions, the fusion protein in such case preferably comprises only one cysteine (C) residue.

The cysteine (C) residue that links the cytotoxic radionuclide to the fusion protein may be a placed in a terminal position. Preferably, the cysteine (C) residue is the C-terminal residue of the fusion protein. In one embodiment, this cysteine (C) residue is the C-terminal residue of an amino acid sequence extending from the C-terminal end of the ABR. Such an amino acid sequence may for example be EEEC (SEQ ID NO:33) or GSSC (SEQ ID NO:34).

Adoptive transfer of immune cells (e.g. T cells, NK cells and macrophages) expressing engineered chimeric antigen receptors (CARs), has recently shown enormous potential for cancer treatment in humans. Adoptive transfer of CD19-directed CAR T cells has generated complete and durable remissions in patients with refractory and relapsed B cell malignancies. The extracellular portion of a CAR comprises an affinity protein directed against a tumor antigen (TA). The affinity protein is typically fused to a transmembrane domain, and intracellular stimulatory domains. Upon binding of the cells to the TA on the cancer cells, endogenous downstream signaling molecules are recruited to transduce signaling, leading to T-cell activation and cancer cell killing. However, in spite of the extraordinary responses observed for B cell malignancies, the potent cell killing and the very long serum circulation time of engineered T cells in the patients (probably life-long), may result in challenges with controlling toxicity in healthy organs and long-term side effects. Accordingly, the treatment is predominantly used for cancers with favorable TA expression profiles. Substantial efforts are therefore focused on strategies for controlling the activity of CAR T cells (see also the review by Krug et al., Cancers (Basel) 2021 Dec. 30; 14(1):183).

The fusion protein of the first aspect may be used in protease-activated CAR immune cells. Such CAR immune cells have the potential to preferentially be active in the protease-containing tumors. Alternatively, the corresponding protease could be co-administered along with the transfusion of CAR immune cells for activation of the cells during a defined time window. After completed treatment, no protease will be available in the patient and the cells will remain inactive. Both strategies have potential to reduce toxicity and long term-side effects and make the treatment available for more cancer forms.

In a variant of the second aspect, a fusion protein of the first aspect is thus expressed on the surface of a cell for use in cell therapy. It follows from the discussion above that the cell may be a T cell, an NK cell or a macrophage, in particular a T cell or an NK cell.

As a third aspect of the present disclosure, there is provided the therapeutic conjugate of the second aspect for use in a therapeutic method of treatment.

The therapeutic method of treatment typically is a method of treatment of a subject suffering from a cancer, such as a cancer overexpressing EGFR. Examples of cancers overexpressing EGFR are lung cancer (especially non-small cell lung cancer), prostate cancer, breast cancer, colon and rectum cancer, head and neck cancer, esophagogastric cancer, liver cancer, glioblastoma, cervix cancer, ovary cancer, bladder cancer, kidney cancer and pancreatic cancer.

In one embodiment, the method of treatment comprises a diagnostic step of quantifying the degree of EGFR expression in the tumor and administration of the therapeutic conjugate in case of overexpression of EGFR in the tumor.

The quantification of the degree of EGFR expression may for example by based on imaging, e.g. using an imaging agent comprising the fusion protein of the first aspect. In such an imaging agent, the fusion protein may be coupled to a radionuclide suitable for imaging. When used for imaging, the fusion protein typically comprises no half-life-extending region.

Subsequent to the administration of the imaging agent comprising the radionuclide, the patient is scanned to detect, visualize and/or quantify EGFR expression. The scanning is typically a tomography, preferably positron emission tomography (PET) or single-photon emission computed tomography (SPECT). For the latter, a CZT-based camera technology may be used.

In an embodiment, the radionuclide suitable for imaging is selected from the group consisting of ¹⁸F, ¹⁴I, ⁷⁶Br, ⁶⁸Ga, ⁴⁴Sc, ⁶¹Cu, ⁶⁴Cu, ⁸⁹Zr, ⁵⁵Co, ⁴¹Ti, ⁶⁶Ga, ⁸⁶Y, ^{110m}In, ¹²³I, ¹³¹I, ^{99m}Tc, ¹¹¹In and ⁶⁷Ga. A preferred group consists of ¹⁸F, ⁶⁸Ga, ^{99m}Tc and ¹¹¹In. Another preferred group consists of ¹⁸F, ⁶⁸Ga and ¹¹¹In.

For radiolabelling with ¹⁸F, a prosthetic group (forming a covalent bond to ¹⁸F) may be coupled to the fusion protein. Examples of resulting structures are N-(2-(4-[¹⁸F]-fluorobenzamido) ethyl) maleimido ([¹⁸F]FBEM), 4-[¹⁸F]-fluorobenzaldehyde ([¹⁸F]-FBA) and [¹⁸F]-fluorophenyloxadiazole methylsulfone ([¹⁸F]-FPOS. Another option is [¹⁸F]aluminium monofluoride in combination with a triaza chelator.

Also in case of radiolabeling with ¹²³I, ¹²⁴I, ¹³¹I and ⁷⁶Br, a prosthetic group may be used. Examples of resulting structures are iodo-/bromo-benzoate and iodo-/bromo-hydroxyphenylethyl mealeimide.

For radiolabeling with ⁶⁸Ga, ⁶⁷Ga, ⁶⁶Ga, ⁴⁴Sc, ⁵⁵Co, ⁴¹Ti, ⁸⁶Y, ^{110m}In and ¹¹¹In, it is preferred to couple a chelator to the HBP. Examples of chelators are DOTA, NOTA, NODAGA and DOTAGA and their derivatives.

For ⁶¹Cu and ⁶⁴Cu, a cross-bridged chelator, such as CB-TE2A, is a better option.

For radiolabelling with ^{99m}Tc, a variety of chelators can be used, such as hexahistidine (H₆) and chelators based on a cysteine- or mercaptoacetyl-containing peptide.

In case of ¹⁸F, ¹²⁴I, ⁷⁶Br, ⁶⁸Ga, ⁴⁴Sc, ⁶¹Cu, ⁶⁴Cu, ⁸⁹Zr, ⁵⁵Co, ⁴¹Ti, ⁶⁶Ga, ⁸⁶Y or ^{110m}In, the scanning technique is preferably PET.

In case of ¹²³I, ¹³¹I, ^{99m}Tc, ¹¹¹In or ⁶⁷Ga, the scanning technique preferably comprises SPECT, e.g. using a CZT-based camera.

The radionuclide is preferably coupled to a terminal end of the fusion protein, such as the C-terminal end of the fusion protein.

In an embodiment, the fusion protein comprises an extension that forms a chelator for the radionuclide. As an example, the chelator-forming part may comprise the sequence HHHHHH (SEQ ID NO:35), which can bind ^{99m}Tc. An alternative to HHHHHH is HEHEHE (SEQ ID NO:30).

As a fourth aspect of the present disclosure, there is provided a method of treatment of a subject suffering from cancer comprising the step of administration of the therapeutic conjugate of the second aspect.

As a fifth aspect of the present disclosure, there is provided a pharmaceutical composition comprising the therapeutic conjugate of the second aspect and a pharmaceutically acceptable carrier.

The composition may for example be adapted for intravenous administration. Accordingly, the composition may be water-based. The water-based composition is preferably buffered, such as phosphate-buffered. As an example, the composition may be based on phosphate-buffered saline. The water-based composition may comprise human serum albumin (HSA). HSA scavenges free radicals and prevents radiolytic damages to the therapeutic conjugate. The amount of HSA may be 10-150 mg/ml, such as 50-100 mg/ml, preferably 75 mg/ml.

### Example 1

### Material and Methods

### Bacterial Culture and Library Expression

S. carnosus cells were grown O/N in B2 medium (i % casein hydrolysate, 2.5% yeast extract, 0.5% D-glucose, 2.5% NaCl, 0.08% K₂HPO₄, pH=7.4) supplemented with 10 μg/mL Chloramphenicol. A combinatorial S. carnosus library expressing 109 different Affibodies variants on the surface was used for the isolation of binders.

### Target Biotinylation

ZEGFR-His₆-Cys was purified by IMAC and biotinylated using EZ-Link™ Maleimide-PEG2-Biotin (Thermo Scientific), according to manufacturer's recommendations. EGFR (His-tag) (Sino Biological Inc., China) was biotinylated using Biotin-XX Microscale Protein Labelling Kit, (Invitrogen, USA) according to manufacturer's recommendations. Absorbance at 280 nm was used to determine the protein concentrations.

### Library Selections Against ZEGFR Using MACS

Streptavidin-coated Dynabeads (Invitrogen, USA) (500 μL) were washed twice with 800 μL of PBS-P (phosphate-buffered saline supplemented with 0.1% Pluronic® F108 NF Surfactant, pH 7.4; BASF Corporation, USA) and incubated with 150 nM of biotinylated ZEGFR for 1 h at RT with gentle rotation. Afterwards, the ZEGFR-coated magnetic beads were washed with PBS-P supplemented with 2 mM EDTA (PBSP-E). A number of cells corresponding to a ten-fold library coverage (10¹⁰ cells) were washed with PBSP-E and the pellet resuspended in PBSP-E to a final concentration of 5·10⁸ cells/mL. The cells were incubated with 1.25 mg of ZEGFR-coated magnetic beads for 1 h at RT with gentle rotation followed by 5 minutes on ice. The tubes were placed on a magnetic rack for 4 minutes to capture the beads and the supernatant was removed. The beads were then resuspended in 30 mL of ice-cold PBS-P. Bead capture and washing was repeated three times. Lastly, the cells were resuspended in 50 mL of B2 media supplemented with 10 μg/mL Chloramphenicol. The cultures were incubated O/N at 37° C. and 150 rpm. Serial dilutions of samples taken before and after the magnetic sorting were used to calculate the population size. Finally, the libraries were incubated with 225 nM Alexa Fluor 647-HSA conjugate and 33.3 nM streptavidin R-phycoerythrin conjugate (SAPE) in PBS-P. The cells were washed twice with ice-cold PBS-P and resuspended in 300 μL of ice-cold PBS-P. The cells were analyzed using a Gallios™ flow cytometer (Beckman Coulter, CA, USA) and the laser protocol used for the detection was FL6-660/20 nm for detection of Alexa Fluor 647-HSA, and FL2-575/20 nm for detection of SAPE.

### Library Selections Against ZEGFR Using FACS

The output cells from the MACS selection (library size≈10⁵ cells) were subsequently sorted using Fluorescence-activated cell sorting (FACS). A 100-fold coverage of the new library size was used for the O/N culture. Cells were washed twice with ice-cold PBS-P and mixed with biotinylated ZEGFR suspended in PBS-P at a concentration of 150 nM. The samples were incubated for 1 h at RT with gentle rotation. Afterwards, the cells were washed twice using ice-cold PBS-P. In order to visualize and sort the cell library by FACS, the cells were incubated with 225 nM Alexa Fluor 647-HSA conjugate and 33.3 nM SAPE on PBS-P. Finally, the cells were washed twice with ice-cold PBS-P and resuspended in 300 μL of ice-cold PBS-P. The cells were subsequently sorted using an Astrios FC cell sorter (Beckman Coulter, USA). The laser protocol used for the detection was 561-585/40 height-log for detection of SAPE, and 640-671/30 height-log for detection of Alexa Fluor 647-HSA. A total of 10⁶ cells were sorted into 1.5 mL B2 media and incubated for 1 h at 37° C. with gentle rotation. Afterwards, the cells were mixed with B2 media supplemented with 10 μg/mL Chloramphenicol to a final volume of 3 mL and incubated at 37° C. O/N with gentle rotation. Finally, the libraries were studied by FC and aliquoted as glycerol stocks to be stored at −80° C. for further experiments.

### Selection and Production of Affibody Candidates

Individual affibody candidates were selected by plating the library on Agar plates supplemented with 10 μg/mL Chloramphenicol and growing single colonies O/N on TSB-Y media (Merck, Germany) supplemented with 10 μg/mL Chloramphenicol. The cells were analysed by FC and sent for sequencing (Microsynth Seqlab, Germany). The coding sequence for the selected candidate Affibody was cloned by restriction cloning into a pETb26+ bacterial expression vector (Addgene, USA). Escherichia coli BL21* cells were transformed with the previously cloned plasmid by standard heat-shock treatment. A single colony from the transformation was inoculated and grown O/N in 10 mL of TSB-Y media supplemented with 50 ng/mL of Kanamycin. After 16 h, the O/N culture was diluted 1:100 in TSB-Y supplemented with 50 ng/mL of Kanamycin to a final volume of 100 mL. The culture was induced with IPTG to a final concentration of 1 mM at OD₆₀₀≈1 and incubated O/N at 27° C. and 200 rpm. After 16 h, the cells were harvested by centrifugation for 8 min at 5000×g and stored at −20° C. The cells were lysed by sonication for 1.5 min (1s ON/1s OFF) followed by centrifugation at 4° C. and 10000×g for 20 min. The supernatant was filtered (0.45 μm) and the protein of interest purified using immobilized metal ion affinity chromatography (IMAC). For this purpose, PD-10 columns packed with 3 ml TALON Metal Affinity Resin. Wash buffer (50 mM Na₂HPO₄, 500 mM NaCl, 15 mM imidazole, pH 8) and elution buffer (50 mM Na₂HPO₄, 500 mM NaCl, 300 mM imidazole, pH 8) were used according to manufacturer's recommendations (GE Healthcare, Sweden). Finally, the buffer was changed to PBS using PD-10 desalting columns according to manufacturer's recommendations (GE Healthcare, Sweden). Purified proteins were analyzed by mass spectrometry (MS) (4800 MALDI TOF/TOF, Applied Biosystems, USA) and SDS-page gels (NuPAGE, Invitrogen, USA).

Evaluation of Secondary Structure. Thermostability and Refolding Capacity of ZB05 Using Circular Dichroism

Circular dichroism spectroscopy was performed using a Chirascan spectropolarimeter (Applied Photophysics, UK) with an optical path length of 1 mm to analyse the alpha-helical content, thermal stability, and refolding capacity of the constructs at a concentration of 0.4 mg/mL. The thermal stability was evaluated by measuring the change in ellipticity at 221 nm during heating (5° C./min) from 20 to 95° C. The melting temperatures (Tm) were approximated from the data acquired from variable temperature measurements (VTM) by curve fitting using a Boltzmann Sigmoidal model (GraphPad Prism version 7, USA). The refolding capacity was assessed by comparing spectra obtained from measurements at wavelengths in the range of 195-260 nm at 20° C., before and after thermal denaturation.

### Affinity Screening by Surface Plasmon Resonance

Target binding was measured for soluble ZB05 Affibody masking candidate and POC-PA molecules using a Biacore 8K SPR instrument (GE Healthcare, Sweden). Approximately 676 RU of ZEGFR and 1000 RU HSA were immobilized by amine coupling on a dextran CM-5 sensor chip according to manufacturer's recommendations (GE Healthcare, Sweden). PBS-T (phosphate-buffered saline supplemented with 0.05% Tween20, pH 7.4) was used as running buffer. The analytes ZB05 and ZEGFR were injected at 5 different concentrations (200, 100, 50, 25 and 12.5 mM) for 150 s followed by dissociation for 150 sec. Intact and cleaved POC-PA were injected at a concentration of 100 nM for 150 s followed by dissociation for 150 sec. The experiments were performed at 25° C. with a flow rate of 100 μL/min. The chips were regenerated by injection of 10 mM HCl for 30 s. The binding kinetics were analysed by the Biacore evaluation software using a Langmuir 1:1 kinetic model.

### Mutagenesis Study of the ZB05 Masking Candidate and Binding Analysis

Each randomized position in the ZB05 sequence were individually mutated to each amino acid except for cysteine, producing a total of 253 sequences. Oligos for each sequence were synthesised and pooled (Twist Bioscience, USA). The pooled oligos were cloned into the S. carnosus display vector pSC2 using NEB Builder (New England Biolabs, USA). The resulting plasmids were transformed into Stellar™ Electrocompetent Cells (Takara Bio, Japan) for amplification and extracted using Qiagen Maxi prep kit (Qiagen, USA). The amplified vectors were subsequently transformed into electrocompetent S. carnosus TM300 cells according to a standard electroporation protocol.

S. carnosus cells were grown O/N in B2 medium supplemented with 10 μg/mL Chloramphenicol. After 16 h, the cells were washed twice with PBS-P and mixed with biotinylated ZEGFR resuspended in PBS-P at a concentration of 150 nM. The samples were incubated for 1 h at RT with gentle rotation. Afterwards, the cells were washed twice using ice-cold PBS-P. To visualize and sort the library by FACS, the cells were incubated with 225 nM Alexa Fluor 647-HSA conjugate and 33.3 nM SAPE in PBS-P. Finally, the cells were washed twice with ice-cold PBS-P and resuspended in 300 μL of ice-cold PBS-P. The cells were subsequently sorted for binding and for non-binding using an Astrios FC cell sorter (Beckman Coulter, USA). The laser protocol used for the detection was 561-585/40 height-log for detection of SAPE, and 640-671/30 height-log for detection of Alexa Fluor 647-HSA. A total of 10⁶ cells per population were sorted into 1.5 mL B2 medium and incubated for 1 h at 37° C. with gentle rotation. Afterwards, the cells were mixed with B2 medium supplemented with 10 μg/mL Chloramphenicol to a final volume of 3 mL and incubated at 37° C. O/N with gentle rotation. Finally, the sorted binding and non-binding populations as well as the naïve library were studied using FC and used for next-generation sequencing.

Deep Sequencing of Libraries from Mutagenesis Study

S. carnosus cells were grown O/N in B2 medium supplemented with 10 μg/mL Chloramphenicol. The Qiagen Miniprep kit was used to purify plasmids from each library populations according to manufacturer instructions (Qiagen, USA). The samples were prepared for deep sequencing by PCR amplifying the plasmids with primers containing the TrueSeq adapters and specific index (Illumina, USA). The sequencing was performed at Scilifelab (National Genomics Infrastructure, Sweden) using a MiSeq 300 cycle instrument (Illumina, USA). The output FASTQ files were analyzed by Geneious version 2020.1.1 (Geneious, New Zealand). Binding and non-binding populations of mutated ZB05 variants as well as the naïve mutagenesis library were sequenced using NGS. The data was normalized to the prevalence of amino acids in each position of the naïve library. Allowed substitutions that retained binding to ZEGFR was determined from a twofold enrichment in the binding population compared to the naïve library with at least a 50% depletion for the corresponding variant in the non-binding population.

POC-PA Sub-Cloning into the Staphylococcal Display Vector

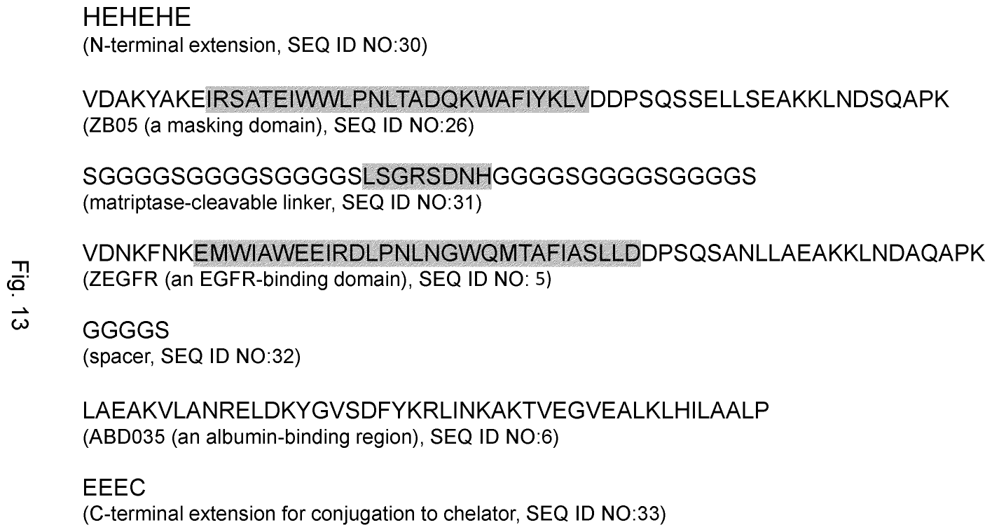

The gene encoding for the EGFR-binding Affibody molecule ZEGFR and the anti-idiotypic Affibody molecule ZB05 were cloned by restriction cloning into a pSC2 vector separated by a TEV protease substrate sequence and linked to an albumin binding protein (ABP). The TEV protease substrate consisted of the sequence ENLYFQG (SEQ ID NO:36) flanked by G₄S repeats in order to extend the length of the linker (the exact sequence of the linker including the TEV protease substrate is shown in FIG. 12). A construct containing an anti-ZHER2 Affibody masking domain (“ZE01”, not binding EGFR) was cloned into pSC2 to be used as control. The resulting plasmid was transformed into E. coli TOP10 for plasmid amplification and subsequently transformed into electrocompetent S. carnosus TM300 cells according to a standard electroporation protocol (Löfblom et al. J. Appl. Microbiol. 2007, 102 (3), 736-747).

The full amino acid of the POC-PA prodrug is shown in FIG. 12.

POC-PA Activation by TEV Protease on S. carnosus Cell Surface

S. carnosus cells displaying the different prodrug constructs were grown O/N following the standard protocol previously described. Approximately 10⁷ bacterial cells (10 μL O/N culture) were washed twice with 800 μL 1×PBS-P and pelleted by centrifugation for 6 min at 4° C. and 6000 rpm. The cells were resuspended either in assay buffer (50 mM Tris-HCl, 0.5 mM EDTA, 1 mM DTT, pH 8) supplemented with 5 Units of TEV protease or in assay buffer only (controls) and incubated for 1 h at 30° C. Bacterial cells were washed three times with PBS-P and incubated in 100 μL of PBS-P supplemented with 50 nM biotinylated EGFR receptor or just PBS-P during 45 min at 37° C. with gentle rotation. Finally, the cells were washed twice with PBS-P and incubated with Alexa Fluor 647-HSA and streptavidin R-phycoerythrin conjugate to be analysed by FC.

### Soluble POC-PA Activation by TEV Protease

The different prodrug constructs were cloned into pET26b+ expression vector and produced in E. coli BL21* cells. The proteins were purified using an automated purification system (ÄXKTA, GE Healthcare, Sweden). For this purpose, a PD-10 desalting column packed with 10 mL HSA-sepharose, 1×TST pH 8, 5 mM, NH4Ac pH 5.5 and 0.5 M HAc was used according to manufacturer's instructions. Proteins were freeze dried and stored at −20° C. The POC-PA (ZB05-TEV_{substrate}−ZEGFR-ABP) protein was resuspended in Assay buffer (50 mM Tris-HCl, 0.5 mM EDTA, 1 mM DTT, pH 8) supplemented with 33 μg of TEV for 1 h at 30° C. The buffer was changed to PBS-0.1% BSA using PD-10 desalting columns according to manufacturer's recommendations (GE Healthcare, Sweden).

### Mammalian Cell Culture

The H292 (human mucoepidermoid pulmonary carcinoma) and A431 (human Squamous cell skin cancer) (American Type Culture Collection, ATCC via LGC Promochem, Sweden) cell lines were used for cell cytotoxicity assays. The cells were cultured in Roswell Park Memorial Institute (RPMI) medium (Flow laboratories, UK) supplemented with 10% fetal bovine serum (Sigma-Aldrich, St. Louis, MO., USA), and a mixture of penicillin 100 IU/mL and 100 μg/mL streptomycin (PEST, Biokrom Kg, Berlin, Germany) (for H292), and Dulbecco's Modified Eagle Medium (DMEM) medium (Flow, Irvine, UK) supplemented with 10% fetal bovine serum (for A431). The cells were grown at 37° C. in a 5% CO₂ atmosphere.

### Flow Cytometric Analysis of EGFR-Binding on H292 and A431 Cells

A431 and H292 cells were cultured according to manufacturer's recommendations (ATCC, USA) and as described above. Trypsin-treated cells (5·10⁵) were resuspended and washed in 500 μL of PBS-0.1% BSA. The cells were incubated in 100 μL of PBS-0.1% BSA supplemented with 100 nM of POC-PA (previously treated with or without TEV protease) for 1 h at RT. The cells were washed once more and incubated with 225 nM Alexa Fluor 647-HSA in PBS-0.1% BSA for 45 min on ice. After a final washing step, the cells were resuspended in 300 μL of PBS-0.1% BSA and analyzed by FC using a Gallios™ flow cytometer (Beckman Coulter, USA).

### Results

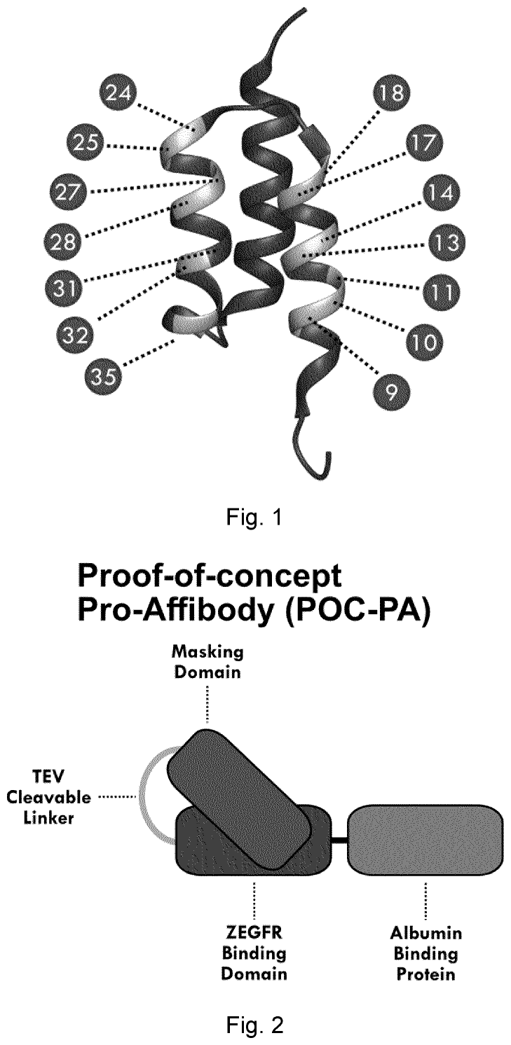

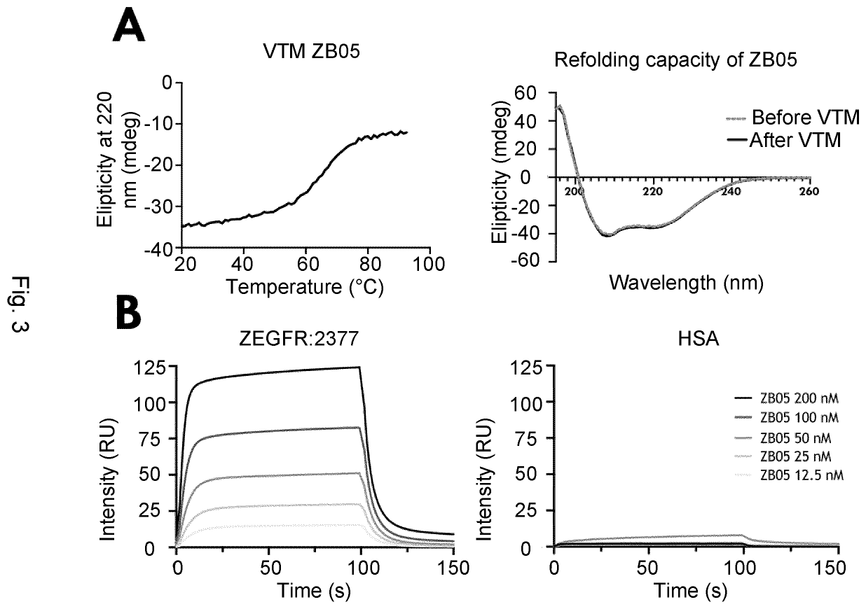

An anti-idiotypic affibody masking domain, denoted ZB05, with specificity for the binding surface of ZEGFR was successfully generated using staphylococcal display by randomization of 14 surface exposed residues (FIG. 1). The purpose of the masking domain is to block the binding of the EGFR-targeting affibody. A proof-of-concept pro-affibody (POC-PA) molecule was constructed consisting of the masking domain fused to the N-terminal of the EGFR-binding affibody molecule ZEGFR via a TEV-protease cleavable linker and an albumin-binding protein (ABP) (FIG. 2 and FIG. 12). The substrate sequence for TEV protease was chosen to facilitate initial characterizations and ABP was included as a purification tag and characterization tool (in a therapeutic application, however, ABP has a half-life-extending effect).

### Isolation of an Affibody Masking Candidate

A naïve combinatorial S. carnosus library with a theoretical size of 109 affibody variants was used to isolate an anti-idiotypic affibody with affinity for the binding surface of the EGFR-binding affibody molecule ZEGFR. Magnetic-assisted cell sorting (MACS) was initially used for two rounds to reduce the complexity of the library prior to several rounds of FACS. The estimated maximal cell diversity after the second MACS selection round was 4·8·10⁵ variants. Three rounds of FACS selection were performed to further enrich the library for binders. In the first round of FACS a 0.42% gate was used to sort cells from the output of the second MACS, creating the library F1A. In the second round of FACS, F1A was sorted using a 3.02% gate resulting in F2A1. Finally, a third round of FACS was performed on the F2A library with a gate encompassing 12.77% of the population resulting in the library F3A1.2. The output from each selection round was analyzed by Flow Cytometry (FC) to confirm enrichment for binding to ZEGFR. Negative controls were used to eliminate the potential for streptavidin-binders (data not shown). The number of putative binders increased after each selection round as seen from flow cytometric analysis of the sorted outputs.

### Affinity Studies of Individual Masking Domain Candidates

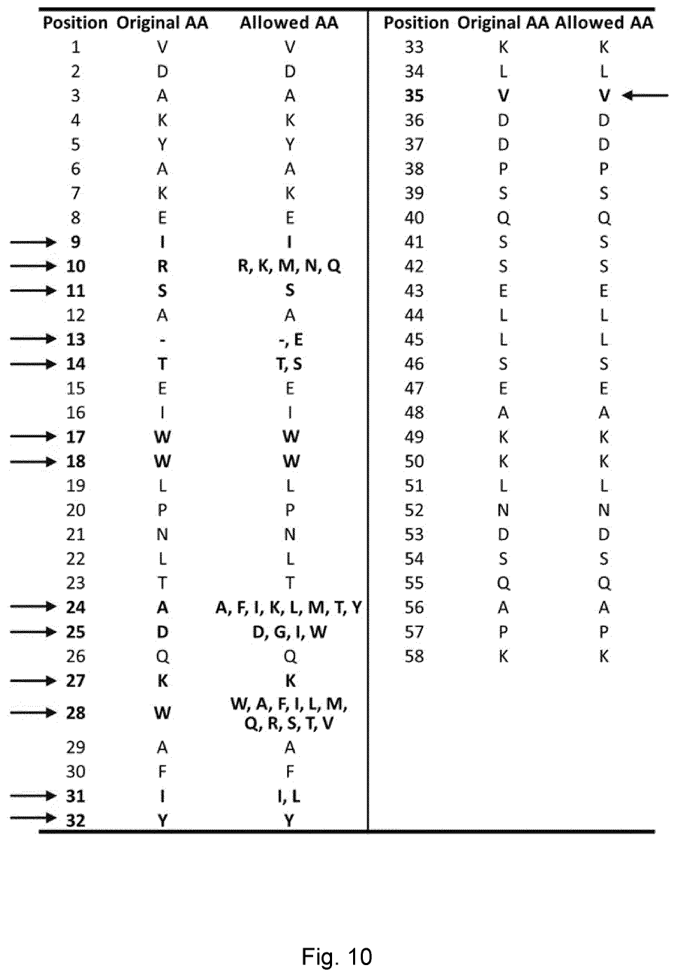

Binding to ZEGFR was analyzed for more than 30 sequenced candidates (both recurring and unique) by FC. One of these candidates (ZB05) was found to exhibit high binding propensity to ZEGFR and was chosen as an anti-idiotypic masking domain candidate for the construction of a pro-affibody targeting EGFR. The sequence of ZB05 is shown in FIG. 10.

### Mutagenesis Study of the ZB05 Masking Candidate and Binding Analysis

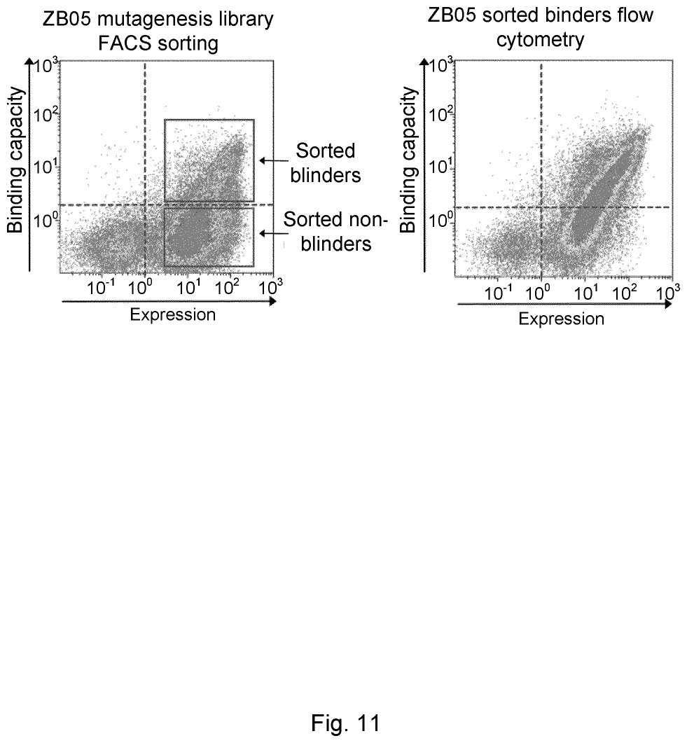

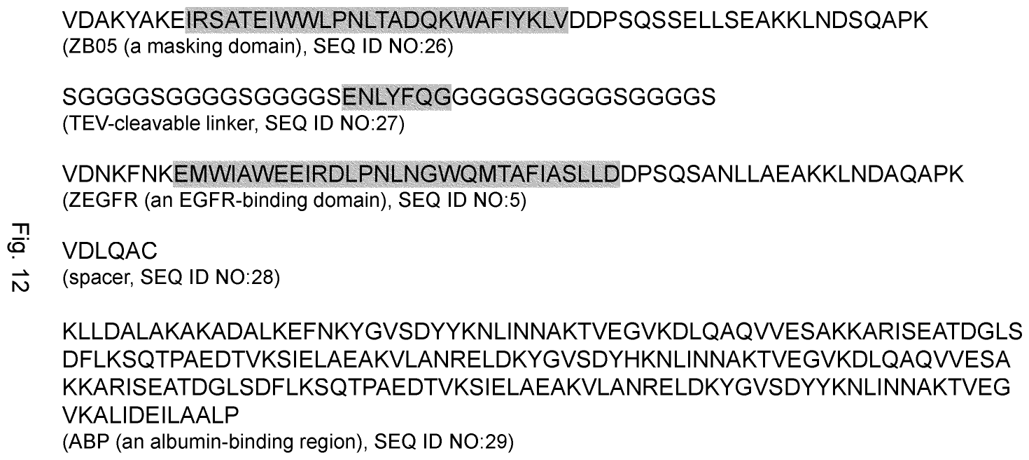

The mutagenesis study of the ZB05 masking domain was performed to evaluate the binding contribution of amino acids in each randomized position and how substituting to any other amino acid affects binding to ZEGFR. The original sequence and the randomized positions of ZB05 are shown in FIG. 10. Binding and non-binding populations of the mutagenesis library, containing 253 different ZB05 variants, were sorted using FACS. Representative sorting of the library and subsequent flow cytometric analysis of the binding population is shown in FIG. 11. Binding and non-binding populations as well as the naïve mutagenesis library were sequenced using NGS and the results are summarized in FIG. 10. Position 13 was included in the mutagenesis library despite the position being deleted in the original ZB05 sequence. From the data, positions that seem to be crucial for binding can be discerned. These include amino acids in positions 9, 11, 17, 18, 27, 32 and 35 where substitution to any other amino acid abrogated binding. Position 14 and 31 have limited flexibility, allowing substitutions from threonine to serine and isoleucine to leucine, respectively. Position 13 was deleted in the original ZB05 sequence and only allows substitution to glutamic acid. The deletion seems to be beneficial for binding, as 40% of the non-binding population contained an amino acid in the deleted position and only 0.6% in the binding population. This is not surprising, as insertion of an amino acid in position 13 would shift the spatial position of amino acids with high apparent binding contribution in position 14, 17 and 18 on helix 1. The remaining positions (10, 24, 25 and 28) are highly variable with several different amino acid substitutions that retain binding.

### Production and Characterization of the Anti-Idiotypic Masking Domain ZB05

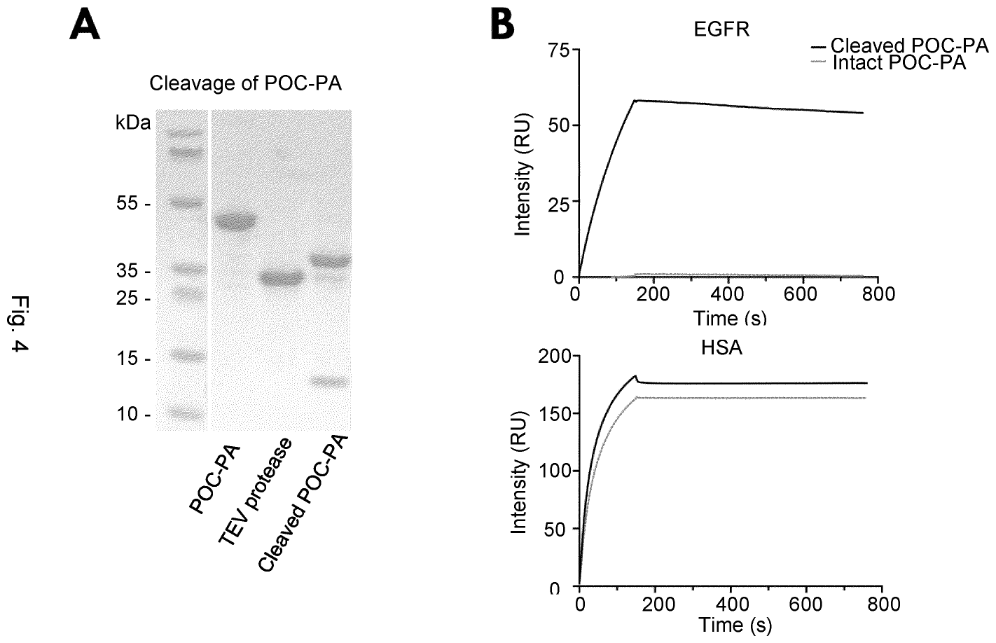

The ZB05 masking candidate was cloned into a pET26b+ vector and produced in E. coli BL21* cells as a soluble monomer containing a C-terminal His-tag, with an expected molecular weight of 7.6 kDa. The protein was purified by IMAC and subsequently analyzed with mass spectrometry (MS) and SDS-page to confirm the mass identity and to evaluate purity, respectively (data not shown). Circular dichroism spectroscopy was used to investigate the secondary structure and determine the thermal stability and refolding capacity. The results from variable temperature measurements (VTM) are shown in FIG. 3 and the melting temperature (Tₘ) was calculated to be 64.1° C. The protein exhibited an expected alpha-helical secondary structure content and was able to completely refold after thermal denaturation (FIG. 3A). A surface plasmon resonance (SPR) based biosensor assay was performed to study the binding affinity of ZB05 to ZEGFR. Five concentrations of ZB05 were injected over a sensor chip with immobilized ZEGFR. HSA was immobilized to screen for unspecific binding. The results confirm binding of ZB05 to ZEGFR with no unspecific binding to the negative control surfaces (FIG. 3B).

Analysis of POC-PA Activity on S. carnosus Cell Surface

To produce a proof-of-concept pro-affibody (POC-PA), both ZB05 and ZEGFR sequences were subcloned into a staphylococcal display vector containing a linker with the TEV substrate coding sequence and a C-terminal albumin binding protein (ABP). The TEV-protease was chosen to facilitate the initial analysis of the interaction between the EGFR-binding ZEGFR and masking ZB05 affibody molecules. In later experiments, the protease substrate sequence could easily be exchanged to accommodate any protease specificity. The POC-PA (ZB05-TEV_{Substrate}−ZEGFR-ABP) was displayed on staphylococci and the binding to recombinant human EGFR was analyzed using FC before and after treatment with TEV-protease. The results demonstrated the masking capacity of ZB05, impeding the binding interaction of ZEGFR with its target, since no binding to EGFR could be detected for the intact POC-PA in this particular experiment (data not shown). However, following treatment with TEV-protease, the binding of ZEGFR to EGFR was restored (data not shown). Overall, the results demonstrated that protease cleavage was a requirement for the POC-PA to bind to EGFR in solution. Furthermore, another construct consisting of an anti-ZHER2 Affibody masking domain and the ZEGFR binding domain (POC-PA-DM) was evaluated in the same conditions to verify that the masking by ZB05 was conferred by the specificity for ZEGFR and not due to steric hindrance or unspecific interactions (data not shown). This result shows that the interaction between the two domains of POC-PA is due to the specific anti-idiotypic binding of ZB05 to ZEGFR.

### Production of Soluble POC-PA and Evaluation of TEV-Cleavage

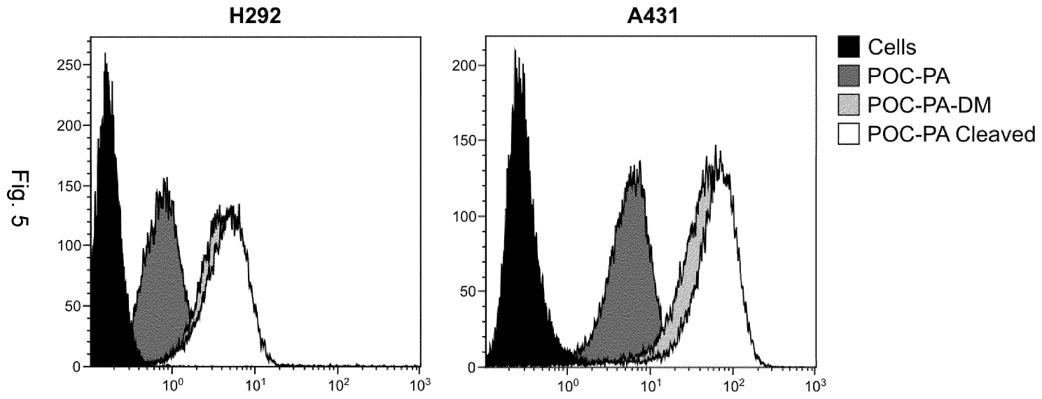

The purified proteins were analyzed both by MS (data not shown) and SDS-page gel to confirm the size and purity of the sample, respectively (FIG. 4A). The protein sample was analyzed before and after incubation with TEV protease, which confirmed cleavage of the prodrug into two products, the ZB05 masking domain (8146 Da) and the ZEGFR-binding domain fused to ABP (30531 Da) (FIG. 4A). The binding of both intact and cleaved POC-PA to immobilized EGFR and HSA was analyzed using SPR. The sensorgrams are shown in FIG. 4B. The results demonstrate the masking capacity of ZB05, which prevents interaction with EGFR for intact POC-PA in this particular experimental set-up. Following cleavage with TEV-protease POC-PA was able to bind EGFR with high affinity. Binding of ABP to HSA was unaffected by the presence of ZB05 or by treatment with TEV-protease. The results corroborate the results obtained from FC, confirming the masking capabilities of ZB05.

### EGFR-Binding Analysis of POC-PA on H292 and 41 Cells Using Flow Cytometry

The binding of POC-PA to native EGFR on the two human cancer cell lines H292 (human mucoepidermoid pulmonary carcinoma) and A431 (human Squamous carcinoma), with moderate and high EGFR-expression respectively, was analyzed for both intact POC-PA and pre-cleaved with TEV-protease (FIG. 5). The construct POC-PA-DM with a dummy (non-ZEGFR binding) masking affibody domain (ZE01) was included to evaluate the influence of steric hindrance on the binding capacity of the EGFR-specific affibody. The signal of the cleaved POC-PA overlaps with the signal for POC-PA-DM in both cell lines and significantly differs from the intact non-cleaved POC-PA, which is indicative of specific blocking of EGFR-binding by the ZB05 masking domain and that protease-mediated activation improves EGFR-binding through the removal of ZB05. However, FIG. 5 also demonstrate that the non-cleaved POC-PA manages to bind the EGFR on the cancer cells, indicating that cleavage of the linker linking the EGFR-binding domain to the masking domain is not necessarily required in a therapeutic application.

### Example 2 (ZEGFR-Based Prodrug Biodistribution)

There are several differences between the prodrug used in this in vivo biodistribution study and the POC-PA prodrug used in Example 1:
In the linker, the TEV-substrate sequence is replaced by a matriptase-substrate sequence;
ABD035 is used for the albumin-binding region (ABR) instead of ABP (ABD035 is a smaller domain with higher affinity);
At the C-terminus, a triglutamyl (EEE) linker followed by a cysteine (SEQ ID NO: 33) for conjugation of the radiometal chelator is added (the triglutamyl (EEE) linker results in increased hydrophilicity);
An N-terminal HEHEHE-tag is added; and
The spacer linking ZEGFR to the ABR is GGGGS (SEQ ID NO:32) instead of VDLQAC (SEQ ID NO:28).

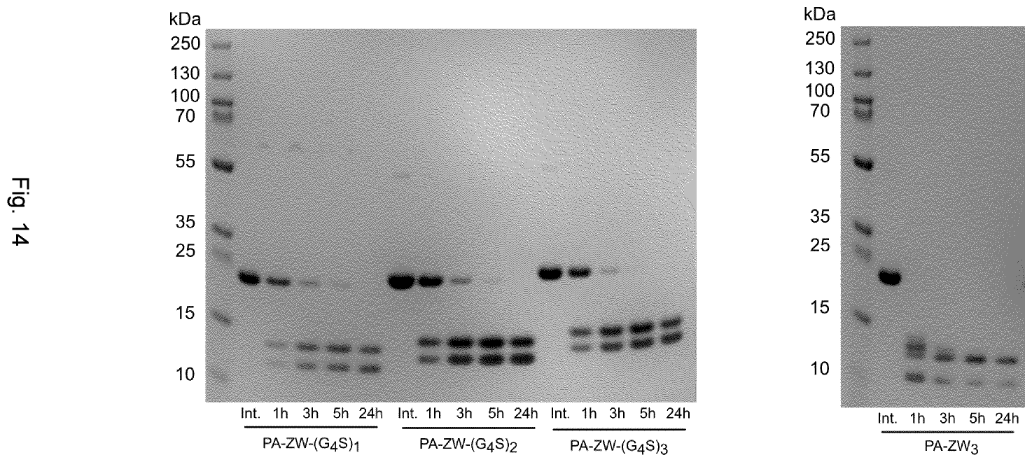

The full amino acid sequence (SEQ ID NO:12) of the prodrug of this Example 2 is shown in FIG. 13.

### Material and Methods

### Cell Culture

Human epidermoid carcinoma cell line A431 (EGFR positive, matriptase low expression), lung mucoepidermoid carcinoma cell line H292 (EGFR positive, matriptase high expression) and lymphoma cell line Ramos (EGFR negative) were obtained from American Type Culture Collection (ATCC, via LGC Promochem, Boris, Sweden). They were maintained in RPMI-1640 medium supplemented with 10% Fetal Bovine Serum (FBS) (Sigma-Aldrich), 2 mM L-glutamine, and a mixture of penicillin 100 IU/mL and 100 μg/mL streptomycin. Cells were grown in a humidified incubator at 37° C. in 5% CO₂ atmosphere.

### Labeling and In Vitro Stability

Indium chloride [¹¹¹In]InCl₃ was purchased from Curium Pharma (Curium Netherlands B.V., Petten, The Netherlands). Buffer were prepared in high quality Mil-Q water, purified from metal contamination using Chelex 100 resin (Bio-Rad Laboratories, Hercules, CA, USA) overnight and filtered 0.2 μm. Three compounds were stored at −20° C. in 0.2 M ammonium acetate, pH 6.0.

Radiolabeling was performed by mixing the compounds (21-25 μg in 88-97 μL 0.2M ammonium acetate, pH 6.0) with [¹¹¹In]InCl₃ (30 MBq in 40 μL 0.05 M HCl). The mixture was thoroughly vortexed and incubated at 80° C. for 60 min. After incubation, 500-fold molar excess of EDTA (160 μg in 16 μL 0.2 M ammonium acetate, pH 6.0) was added and the mixture was incubated at 80° C. for 10 min. The reaction mixture was then purified using NAP-5 size exclusion column pre-equilibrated and eluted with phosphate-buffered saline (PBS). Radiochemical yield and purity of the compounds were determined with iTLC eluted with 0.2 M citric acid, pH 2.0. In this system, the [¹¹¹In]In-labeled compounds stay in application point while free [¹¹¹In]In moves with the solvent front. Distribution of the activity among the strips was measured using a Storage Phosphor System (CR-35 BIO Plus, Elysia-Raytest, Bietigheim-Bissingen, Germany) and analyzed using AIDA Image Analysis software (Elysia-Raytest, Bietigheim-Bissingen, Germany).

In vitro stability was evaluated in PBS and in the presence of 1000-fold molar excess of ethylenediaminetetraacetic acid (EDTA). After purification, samples of labeled compounds (1-1.6 μg, 50 μL in 1% BSA in PBS) were mixed with EDTA (20 μg, 2 μL in PBS) and incubated at room temperature for up to 48 h. Samples were taken for iTLC analysis at i h, 4 h, 24 h and 48 h.

### Binding Specificity in EGFR-Expressing Cells In Vitro

An in vitro binding specificity test was performed according to previously described method (Wällberg et al., Cancer Biother Radiopharm. 2008; 23(4):435-42). In brief, EGFR-positive A431 and H292 cells were seeded in 6-well plates with a density of 10⁶ cells/well one day before the experiment. During the experiment day, a solution of radiolabeled compounds (10 nM) was added to the cell plates. For blocking, 1000 nM of non-labeled ZEGFR-ABD035-DOTA was added 15 min before the radiolabeled compounds to saturate the receptors in a set of (n=3) wells at room temperature. Same volume of culture medium was added to another set of dishes to compensate the volume. The cells were incubated at 37° C. for 60 min. Thereafter, the medium was removed, and the cells were washed with 1 mL PBS wash. The cells were then detached by a trypsin-EDTA solution and collected. The cell-associated activity was measured using an automated γ-spectrometer (2480 Wizard; Wallac, Finland).

An additional in vitro binding experiment was performed to compare EGFR expression levels in H292 and A431 in vitro. Cells were incubated with the radiolabeled ZEGFR-ABD035-DOTA (10 nM), without blocking, and with blocking with cetuximab (1000 nM) or non-labelled ZEGFR-ABD035-DOTA (1000 nM) as described above. The same amount of radioactivity (1.64×10⁶ counts per minute (CPM)) was added to each cell culture dish. Additionally, the cells were counted in additional culture dishes (n=3) for each cell line. The specific cell bound activity was calculated as CPM/10⁶ cells for non-blocked cells minus CPM/10⁶ cells blocked with cetuximab.

### Animal Studies

The animal experiments were approved by the Local Ethics Committee for Animal Research in Uppsala.

Female NMRI mice and Balb/c nu/nu mice were supplied from Scanbur A/S (Karlslunde, Denmark) and had an adaptation period of one week before the start of experimental procedures.

### Biodistribution in NMRI Mice

To test if masking of the binding site prevents hepatic uptake, the biodistribution study was performed at 4 h and 24 h post-injection in 24 female NMRI mice. For each time point, a group of four mice was intravenously (i.v.) injected with equimolar amount (133 μmol) of respective compounds: 3 μg of [¹¹¹In]In-labeled prodrug, 3 μg of [¹¹¹In]In-labeled dummy-linker or 1.8 μg of [¹¹¹In]In-labeled non-masked control (40 kBq in 100 μL 1% BSA in PBS per mouse). In the prodrug, the linker (linking ZB05 to ZEGFR) was based on the sequence LSGRSDNH (SEQ ID NO:21), which was extended on both sides by repeats of G₄S (the exact sequence of this linker is shown in FIG. 13). Hence the total length of this linker was 39 amino acid residues. LSGRSDNH is recognized by e.g. matriptase and hence protease-cleavable. In the non-cleavable variant thereof (i.e. “dummy-linker”), the linker was instead two repeats of GGGS (SEQ ID NO:37) extended on both sides by three repeats of GGGGS (SEQ ID NO:32). The total length of the non-cleavable linker was thus also 39 amino acid residues.

The mice were euthanized by an overdose of anesthesia solution (30 μl of solution per gram body weight, ketamine 10 mg/mL and xylazine 1 mg/mL) and sacrificed by heart puncture. Blood, salivary gland, lung, liver, spleen, pancreas, small intestine, large intestine, kidney, muscle, bone were collected and weighed. Gastrointestinal tract (with its content) and remaining carcass were also collected. The activity of the organs and standards of injected solution was measured using an automated γ-spectrometer (2480 Wizard; Wallac, Finland). The uptake values were calculated as percentage of injected dose per gram sample weight (% ID/g) except for the gastrointestinal tract (with its content) and remaining carcass, which was calculated as percentage injected dose (% ID) per whole sample.

### Implantation

For H292 tumor implantation, female Balb/c nu/nu mice were subcutaneously injected with 10×10⁶ H292 cells in 100 μL of culture medium 1:1 mixed with Matrigel in the right hind leg. The experiments were performed 13-16 days after the implantation. For Ramos implantation, female Balb/c nu/nu mice were subcutaneously injected with 5×10⁶ Ramos in 100 μL of culture medium in the left hind leg. The experiments were performed 13-15 days after the implantation. For A431 tumor implantation, female Balb/c nu/nu mice were subcutaneously injected with 10×10⁶ A431 cells in 100 μL of culture medium in the left hind leg. The experiments were performed 16 days after the implantation. The average animal weight was 17.6±1.3 g in the H292 groups, 18.6±1.3 g in the Ramos groups and 17.3±1.8 g in the A431 groups. The average tumor weight was 0.38±0.23 g for H292 xenografts, 0.19±0.11 g for Ramos xenografts and 0.11±0.06 g for A431 xenografts.

To evaluate the biodistribution of 3 compounds over time, 24 mice bearing H292 xenografts were randomized into 6 group (n=4). Each two groups were i.v. injected with 133 μmol of [¹¹¹In]In-labeled Prodrug, [¹¹¹In]In-labeled dummy-linker or [¹¹¹In]In-labeled non-masked control (40 kBq in 100 μL 1% BSA in PBS per mouse), respectively. Organs and tumors were collected at 4 h and 48 h post-injection, weighed and measured for activity as described above.

In Vivo Specificity of EGFR Binding (H292 vs. Ramos 4 h and 48 h)

To evaluate the in vivo specificity, 8 mice bearing EGFR-positive H292 xenografts and 8 mice bearing EGFR-negative Ramos xenografts were injected with 133 μmol of [¹¹¹In]In-labeled Prodrug. Organs and tumors were collected at 4 h and 48 h p.i., weighed and measured for activity as described above.

### In Vivo Study of Matriptase Function (H292 and A41 in Same Animals. 48 h)

To study the relationship between tumor uptake of Prodrug and matriptase level, 5 mice bearing H292 xenografts (EGFR positive, matriptase high expression) in the right hind leg and A431 xenografts (EGFR positive, matriptase low expression) in the left hind legs were i.v. injected with 133 μmol of [¹¹¹In]In-labeled Prodrug (40 kBq in 100 μL 1% BSA in PBS per mouse). Mice were euthanized at 48 h p.i., organs, half of the liver and partially of the A431 and H292 xenografts were collected, weighted and measured for activity as described above. Another half of the liver and partially A431 and H292 xenografts were collected, formalin fixed and embedded in paraffin for histopathologic exam.

### Imaging (H292 and Ramos, 4 h 48 h)

To image the EGFR expression, whole-body SPECT/CT scans were performed using a nanoScan SPEC/C (Mediso Medical Imaging Systems, Budapest, Hungary). One mouse bearing H292 xenograft was i.v. injected with [¹¹¹In]In-labeled Prodrug (14.7 μg, 1.9 MBq). As a control, one mouse bearing H292 xenograft was i.v. injected with [¹¹¹In]In-labeled non-masked control (8.8 μg, 1.1 MBq). To clarify the in vivo specificity, one mouse bearing Ramos xenograft was i.v. injected with [¹¹¹In]In-labeled Prodrug (11.2 μg, 2.9 MBq). The mice were imaged at 4 h and 48 h p.i. Imaging of [¹¹¹In]In-labeled labeled compounds and image reconstruction were performed as described earlier (Rinne et al. J. Mol Sci. 2020 Feb. 15; 21(4):1312).

### Statistical Analysis

Statistical analysis was performed using GraphPad Prism (version 9.0.0; GraphPad Software, Inc., La Jolla, CA, USA). The in vitro and in vivo data were analyzed using an unpaired two-tailed t-test. A p value <0.05 was considered a statistically significant difference.

### Results

### Labeling and Stability

Radiolabeling of three compounds with indium-111 was performed in radiochemical yield ranged from 10% to 21%. Purification after size exclusion column provided over 97% purity (Table 1). No release of activity during incubation with an excess of EDTA was observed (Table 2).

TABLE 1
Results of indium-111 labeling of Prodrug,
Dummy-linker and Non-masked control.

| | Radiochemical | Radiochemical |
|---|---|---|
| | Yield, % | Purity, % |
| Compound | (n = 3) | (n = 3) |
| ¹¹¹In-Prodrug | 16 ± 3 | 98 ± 1 |
| ¹¹¹In-Dummy-linker | 11 ± 1 | 98 ± 1 |
| ¹¹¹In-Non-masked control | 17 ± 4 | 98 ± 1 |

TABLE 2
In vitro stability of [¹¹¹In]In-labeled Prodrug, Dummy-linker and Non-masked
control. Indium-labeled compounds were incubated at room temperature for 48
h with 1000-fold molar excess of EDTA. Analysis was performed in duplicates.

| | | Protein-Associated Activity (%) | | | | | | | |
|---|---|---|---|---|---|---|---|---|---|
| | | 1 h | | 4 h | | 24 h | | 48 h | |
| | Purity | 1000x | | 1000x | | 1000x | | 1000x | |
| Compounds | (%) | EDTA | PBS | EDTA | PBS | EDTA | PBS | EDTA | PBS |
| ¹¹¹In- | 100 | 99 ± 1 | 99 ± 1 | 99 ± 0 | 99 ± 0 | 98 ± 0 | 99 ± 0 | 98 ± 0 | 99 ± 0 |
| prodrug | | | | | | | | | |
| ¹¹¹In- | 100 | 100 ± 0 | 99 ± 0 | 99 ± 0 | 99 ± 0 | 98 ± 0 | 99 ± 0 | 97 ± 1 | 99 ± 0 |
| dummy- | | | | | | | | | |
| linker | | | | | | | | | |
| ¹¹¹In-non- | 100 | 100 ± 0 | 100 ± 0 | 99 ± 1 | 100 ± 0 | 99 ± 0 | 100 ± 0 | 99 ± 0 | 99 ± 1 |
| masked | | | | | | | | | |
| control | | | | | | | | | |

### In Vitro Studies

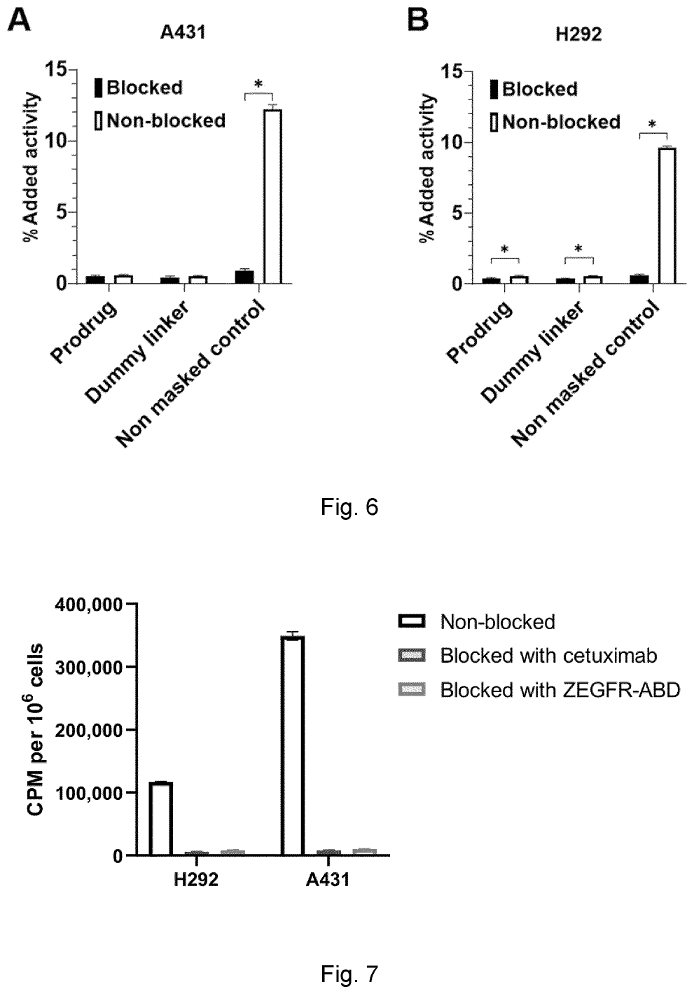

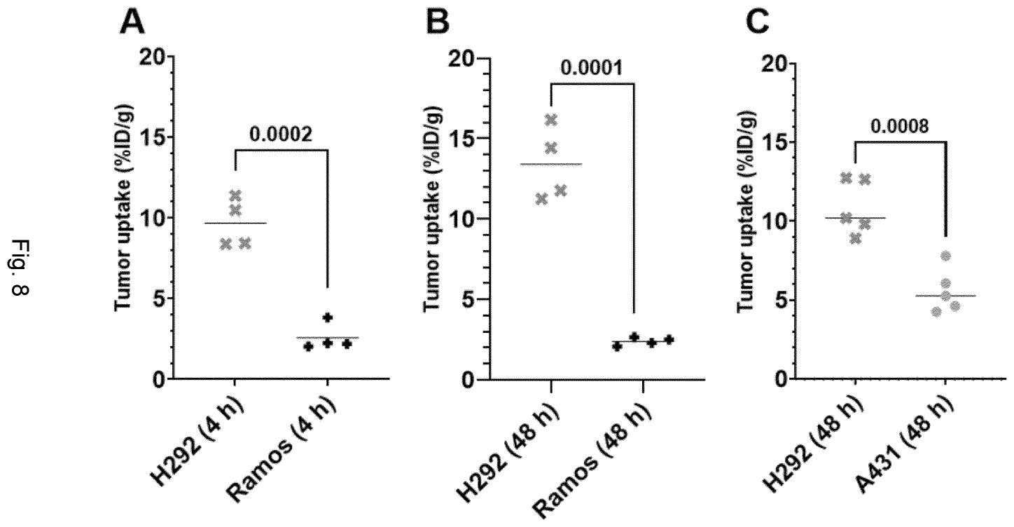

Binding specificity test was performed with A431 and H292 cell lines. Both cell lines over-express EGFR. A significant (p<0.05, t-test) reduction of activity was observed for Non-masked control in the blocked group for both cell lines. This confirmed EGFR-mediated binding of Non-masked control to A431 and H292 cells. No significant difference was observed for prodrug or dummy-linker between blocked and non-blocked groups in A431 cell line (FIG. 6A). However, a small but significant (p<0.05, t-test) difference was observed for prodrug or dummy-linker between blocked and non-blocked groups in H292 cell line (FIG. 6B). Specific binding of [1¹In]In-labeled non-masked control to A431 cell was 3-fold higher than to H292 cells (FIG. 7), which suggest that the EGFR expression by this cell line is also 3-fold higher.

### Biodistribution in NMRI Mice

Data concerning biodistribution of the three compounds in NMRI mice are presented in Table 3. The activity concentration in blood was over 15% ID/g at 4 h p.i. and over 6% ID/g at 24 h p.i for all three compounds. For comparison, the blood concentration of non-ABD035-fused ¹¹¹In-ZEGFR was 0.34*0.02 and 0.12*0.03% ID/g, at 4 and 24 h, respectively (Tolmachev et al. Eur J Nucl Med Mol Imaging. 2010 March; 37(3):613-22). This indicates that fusion to ABD035 results in prolongation of circulation time. The biodistribution of [¹¹¹In]In-labeled Prodrug and Dummy-linker was very similar, except a small but significant (P<0.05, t-test) different kidney uptake at 4 h p.i., as well as significant different of salivary gland uptake and liver uptake at 24 h p.i. Both [¹¹¹In]In-labeled prodrug and dummy-linker had significantly lower liver uptake than Non-masked control at 4 h and 24 h p.i.

TABLE 3
Biodistribution of three [¹¹¹In]In-labeled compounds in NMRI
mice at 4 and 24 h post-injection. The uptake of activity is
expressed as % of injected dose per gram tissue (% ID/g). Results
are presented as an average value from four animals ± 1 SD.

Uptake, % ID/g

| | Prodrug | Dummy-linker | Non-masked |
|---|---|---|---|
| | 4 h | | |
| Blood | 18.7 ± 2.3 | 21.8 ± 1.3 | 19.5 ± 1.0 |
| Salivary gland | 2.9 ± 0.9 | 3.7 ± 1.9 | 2.1 ± 0.4 |
| Lung | 6.3 ± 1.0 | 6.3 ± 2.0 | 5.8 ± 0.3 |
| Liver | 4.0 ± 0.5 | 4.6 ± 0.3 | 17.2 ± 1.3 |
| Spleen | 2.9 ± 0.5 | 3.3 ± 0.3 | 3.3 ± 0.4 |
| Pancreas | 1.6 ± 0.2 | 1.6 ± 0.5 | 1.3 ± 0.3 |
| Small intestine | 2.3 ± 0.6 | 2.6 ± 0.7 | 3.0 ± 1.2 |
| Large intestine | 1.8 ± 0.2 | 1.9 ± 0.5 | 1.7 ± 0.7 |
| Kidney | 10.8 ± 1.5 | 13.5 ± 1.3 | 10.2 ± 0.7 |
| Muscle | 1.3 ± 0.1 | 1.3 ± 0.1 | 0.8 ± 0.1 |
| Bone | 2.2 ± 0.2 | 2.2 ± 0.2 | 2.0 ± 0.1 |
| | 24 h | | |
| Blood | 8.9 ± 1.5 | 10.5 ± 1.1 | 9.6 ± 1.4 |
| Salivary gland | 2.6 ± 0.5 | 3.4 ± 0.3 | 3.2 ± 0.5 |
| Lung | 3.7 ± 0.8 | 4.4 ± 0.4 | 3.8 ± 0.7 |
| Liver | 3.7 ± 0.5 | 4.6 ± 0.4 | 12.7 ± 1.8 |
| Spleen | 2.7 ± 0.7 | 3.4 ± 0.6 | 3.2 ± 0.6 |
| Pancreas | 1.4 ± 0.4 | 1.8 ± 0.2 | 1.9 ± 0.1 |
| Small intestine | 1.7 ± 0.5 | 1.9 ± 0.7 | 3.5 ± 1.1 |
| Large intestine | 1.2 ± 0.3 | 1.4 ± 0.2 | 1.8 ± 0.3 |
| Kidney | 8.7 ± 1.8 | 10.7 ± 1.5 | 7.9 ± 1.2 |
| Muscle | 1.3 ± 0.2 | 1.5 ± 0.1 | 1.1 ± 0.1 |
| Bone | 1.9 ± 0.4 | 2.0 ± 0.2 | 1.9 ± 0.3 |

### Biodistribution in Balb/c Nu/Nu Mice Bearing H292 Xenograft

To evaluate the tumor uptake over time, mice bearing H292 xenografts were injected with [¹¹¹In]In-labeled compounds. Data concerning biodistribution at 4 h and 48 h p.i. are presented in Table 4. Equal levels (p>0.3, t-test) of tumor uptake at both time points for three compounds was observed. [¹¹¹In]In-labeled prodrug and dummy-linker had significantly (p<0.05, t-test) higher blood activity, higher uptake in organs such as: lung, kidney and muscle and significantly (p<0.05, t-test) lower liver uptake than [¹¹¹In]In-labeled non-masked control at both 4 h and 48 h p.i.

TABLE 4
Biodistribution in Balb/c nu/nu mice bearing H292 xenografts,
4 h and 48 h post-injection. The uptake of activity is presented
as percentage of injected dose per gram tissue (% ID/g). Results
are presented as an average value from four animals ± 1 SD.

Uptake, % ID/g

| | Prodrug | Dummy-linker | Non-masked |
|---|---|---|---|
| | 4 h | | |
| Blood | 27.6 ± 2.7 | 30.3 ± 3.2 | 19.3 ± 1.1 |
| Salivary gland | 3.2 ± 0.7 | 3.8 ± 0.6 | 2.3 ± 0.6 |
| Lung | 9.2 ± 0.8 | 11.0 ± 0.6 | 6.8 ± 1.6 |
| Liver | 6.1 ± 0.5 | 7.3 ± 0.9 | 36.7 ± 6.8 |
| Spleen | 4.7 ± 0.5 | 4.8 ± 0.4 | 4.2 ± 1.0 |
| Pancreas | 1.6 ± 0.2 | 2.1 ± 0.5 | 1.3 ± 0.1 |
| Small intestine | 3.0 ± 0.7 | 4.4 ± 1.1 | 3.3 ± 0.6 |
| Large intestine | 1.9 ± 0.1 | 2.4 ± 0.2 | 1.7 ± 0.1 |
| Kidney | 16.6 ± 0.2 | 19.3 ± 1.9 | 11.7 ± 1.8 |
| Tumor | 9.7 ± 1.5 | 10.3 ± 1.3 | 11.4 ± 3.2 |
| Muscle | 1.0 ± 0.1 | 1.2 ± 0.1 | 0.8 ± 0.1 |
| Bone | 2.1 ± 0.3 | 2.6 ± 0.6 | 1.8 ± 0.3 |
| | 48 h | | |
| Blood | 10.3 ± 0.9 | 9.5 ± 0.8 | 4.6 ± 0.6 |
| Salivary gland | 3.9 ± 0.3 | 4.0 ± 0.2 | 3.1 ± 0.5 |
| Lung | 5.6 ± 0.5 | 5.2 ± 0.2 | 2.8 ± 0.5 |
| Liver | 8.8 ± 1.0 | 7.4 ± 0.4 | 16.4 ± 3.0 |
| Spleen | 7.5 ± 0.8 | 6.8 ± 0.6 | 5.5 ± 1.2 |
| Pancreas | 1.7 ± 0.2 | 1.6 ± 0.1 | 1.3 ± 0.4 |
| Small intestine | 2.4 ± 0.5 | 2.4 ± 0.1 | 2.8 ± 0.7 |
| Large intestine | 1.7 ± 0.2 | 1.6 ± 0.2 | 1.6 ± 0.4 |
| Kidney | 13.3 ± 1.1 | 13.6 ± 0.6 | 7.6 ± 1.8 |
| Tumor | 13.4 ± 2.3 | 12.2 ± 1.4 | 13.4 ± 2.6 |
| Muscle | 1.4 ± 0.1 | 1.6 ± 0.5 | 0.7 ± 0.1 |
| Bone | 2.3 ± 0.2 | 2.3 ± 0.1 | 1.3 ± 0.3 |

### In Vivo Specificity

To determine the in vivo EGFR binding specificity, biodistribution was compared in mice bearing H292 xenografts and Ramos xenografts at 4 h and 48 h p.i. At both time points, EGFR-positive H292 xenograft has significantly (p<0.05, unpaired t-test) higher Prodrug uptake than EGFR-negative Ramos xenograft (FIG. 8A, 8B). The uptake in H292 xenografts was 10±2% ID/g and 13±2% ID/g while uptake in Ramos xenografts was 3±1% ID/g and 2±0% ID/g, at h 4 and 48 h p.i., respectively.

To evaluate the uptake of Prodrug in the presence of matriptase, biodistribution was compared in mice bearing both H292 and A431 xenografts at 48 h p.i. The uptake in matriptase-positive H292 xenograft (11±2% ID/g) was significantly (p<0.05, paired t-test) higher than in matriptase-negative A431xenografts (6±1% ID/g) (FIG. 8C).

### Imaging

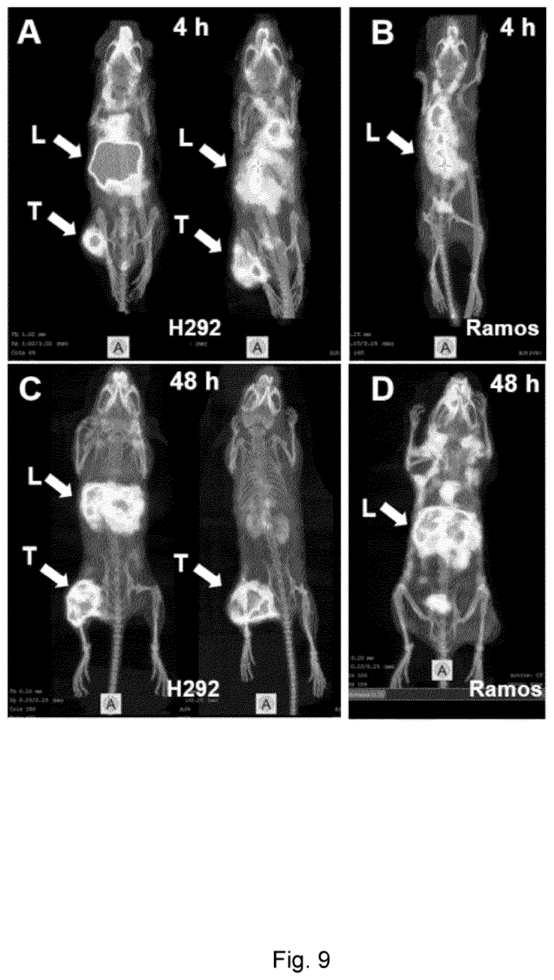

The biodistribution data were confirmed by experimental microSPECT/CT imaging for [¹¹¹In]In-labeled Prodrug and Non-masked control in H292 xenografts (FIGS. 9A, 9C) and [¹¹¹In]In-labeled Prodrug in Ramos xenografts (FIGS. 9B, 9D). Imaging enabled clear visualization of EGFR-expressing H292 xenografts with both [¹¹¹In]In-labeled Prodrug and Non-masked control at 4 h and 48 h p.i., but Ramos xenograft was not visible after injection of [¹¹¹In]In-labeled Prodrug at either time point.

### Example 3 (ZEGFR-Based Prodrug Biodistribution)

Four new pro-affibody (PA) variants were designed to improve the cleavage efficiency by matriptase. A new protease recognition sequence (MSGRSANA, SEQ ID NO:38, “ZW”) developed by the company Zymeworks was used for PA-ZW-(G₄S)₁, PA-ZW-(G₄S)₂, PA-ZW-(G₄S)₃ and PA-ZW₃, replacing the original LSGRSDNH sequence used above:
PA-ZW-(G₄S)₁: (HE)₃-ZB05-(G₄S)-(ZW)-(G₄S)-ZEGFR-ABD035-EEEC; PA-ZW-(G₄S)₂: (HE)₃-ZB05-(G₄S)₂-(ZW)-(G₄S)₂—ZEGFR-ABD035-EEEC; PA-ZW-(G₄S)₃: (HE)₃-ZB05-(G₄S)₃-(ZW)-(G₄S)₃—ZEGFR-ABD035-EEEC; and PA-ZW₃: (HE)₃-ZB05-ZW₃—ZEGFR-ABD035-EEEC.

In the new PAs, the linker interconnecting ZB05 and ZEGFR thus contains a single ZW sequence flanked by 1-3 G4S sequences or a concatemer of three ZW substrate sequences.

The new variants were produced and purified using affinity chromatography, freeze-dried and dissolved in PBS. Four identical samples were prepared for each variant. Each sample contained 10 uM of PA protein and 20 nM of recombinant human matriptase (cat. Nr. 3946-SEB) in a total volume of 50 uL PBS. The samples were incubated at 37 degrees Celsius for 1, 3, 5 or 24 hours followed by freezing at −20 degrees Celsius. The frozen samples were thawed and analyzed with SDS-PAGE, which included the intact (int.) non-cleaved protein from the same stock used for sample preparation.

As shown in FIG. 14, efficient matriptase cleavage was observed for the variants with the ZW substrate sequence. Close to complete cleavage for variants with a single ZW sequence can be seen after 5 hours. Complete cleavage for the concatemer containing the ZW₃ sequence can be seen after only 1 hour.

1. A fusion protein comprising an EGFR-binding domain, a masking domain and a linker linking the masking domain to the EGFR-binding domain, wherein:
the masking domain comprises the amino acid sequence
IX₁₀SX₁₂X₁₃X₁₄X₁₅X₁₆WWX₁₉X₂₀X₂₁X₂₂X₂₃X₂₄X₂₅X₂₆KX₂₈X₂₉X₃₀X₃₁Y
X₃₃X₃₄V
wherein, independently of each other,
X₁₀ is R, K, M, N or Q;
X₁₂ is A or a substitution;
X₁₃ is E or absent;
X₁₄ is T or S;
X₁₅ is E or a substitution;
X₁₆ is I or a substitution;
X₁₉ is L, a substitution or absent;
X₂₀ is P, a substitution or absent;
X₂₁ is N, a substitution or absent;
X₂₂ is L, a substitution or absent;
X₂₃ is T or a substitution;
X₂₄ is A, F, I, K, L, M, T or Y;
X₂₅ is D, G, I or W;
X₂₆ is Q or a substitution;
X₂₈ is W, A, F, I, L, M, Q, R, S, T or V;
X₂₉ is A or a substitution;
X₃₀ is F or a substitution;
X₃₁ is I or L;
X₃₃ is K or a substitution; and
X₃₄ is L or a substitution,
provided that no more than six of X₁₂, X₁₅, X₁₆, X₁₉, X₂₀, X₂₁, X₂₂, X₂₃, X₂₆, X₂₉, X₃₀, X₃₃ and
X₃₄ are substitutions and that no more than two of X₁₉-X₂₂ are absent; and
the EGFR-binding domain comprises the amino acid sequence
EX₂X₃X₄AX₆X₇EIX₁₀X₁₁LPNLNX₁₇X₁₈QX₂₀X₂₁AFIX₂₅SLX₂₈D,
wherein, independently of each other,
X₂ is M, F, V, L, I or S;
X₃ is W, D, E or L;
X₄ is I, V, G, S, M, L, A, T, N, D or W;
X₆ is W, V, L, I, M or S;
X₇ is D, E, N or K;
X₁₀ is R, G, H or K;
X₁₁ is D, N, E, Y or S;
X₁₇ is G, W or A;
X₁₈ is W, G or A;
X₂₀ is M, L, F, A or E;
X₂₁ is T, D, N, A or Q;
X₂₅ is A, S, N, G or L; and
X₂₈ is L, W, V, F or A.

2. The fusion protein of claim 1, wherein, in the masking domain, no more than four of X₁₂, X₁₅, X₁₆, X₁₉, X₂₀, X₂₁, X₂₂, X₂₃, X₂₆, X₂₉, X₃₀, X₃₃ and X₃₄ are substitutions and/or no more than one of X₁₉-X₂₂ is absent.

3. The fusion protein of claim 2, wherein, in the masking domain, no more than two of X₁₂, X₁₅, X₁₆, X₁₉, X₂₀, X₂₁, X₂₂, X₂₃, X₂₆, X₂₉, X₃₀, X₃₃ and X₃₄ are substitutions and/or none of X₁₉-X₂₂ is absent.

4. The fusion protein of claim 1, wherein, in the masking domain, X₁₀ is R, X₁₃ is absent, X₁₄ is T, X₂₄ is A, X₂₅ is D, X₂₈ is W and/or X₃₁ is I.

5. The fusion protein of claim 1, wherein the masking domain comprises the amino acid sequence
IRSX₁₂TX₁₅X₁₆WWX₁₉X₂₀X₂₁X₂₂X₂₃ADX₂₆KWX₂₉X₃₀IYX₃₃X₃₄V.

6. The fusion protein of claim 1, wherein the masking domain comprises the amino acid sequence
(SE ID NO: 1)
IRSATEIWWLPNLTADQKWAFIYKLV.

7. The fusion protein of claim 1, wherein, in the EGFR-binding domain, X₃ is W, X₆ is V or W, X₁₀ is R or G, X₁₇ is W or G, X₁₈ is W or G and/or X₂₁ is T or D.

8. The fusion protein of claim 1, wherein the EGFR-binding domain comprises the amino acid sequence
EX₂WX₄AWX₇EIRX₁₁LPNLNGWQX₂₀TAFIX₂₅SLX₂₈D,
wherein, independently of each other,
X₂ is M, V, L or I;
X₄ is I, V, G, S, M, L, A, T, N or D;
X₇ is D, E, N or K;
X₁₁ is D, N, E, Y or S;
X₂₀ is M, L or F;
X₂₅ is A, S, or G; and
X₂₈ is L or V.

9. The fusion protein of claim 1, wherein, in the EGFR-binding domain, X₂ is M, X₄ is I, V, G or S, X₁₁ is D, N or E, X₂₀ is M, X₂₅ is A or S and/or X₂₈ is L.

10. The fusion protein of claim 1, wherein the EGFR-binding domain comprises the amino acid sequence
(SEQ ID NO: 2)
EMWIAWEEIRDLPNLNGWQMTAFIASLLD.

11. The fusion protein of claim 1 further comprising a half-life-extending region.

12. The fusion protein of claim 1, wherein the linker is a protease-cleavable linker.

13. The fusion protein of claim 12, wherein the protease-cleavable linker comprises a sequence selected from the group consisting of GFLG (SEQ ID NO:11), Glutamic acid-Valine-Citrulline, GILGVP (SEQ ID NO:13), GPLGIAGQ (SEQ ID NO:14), VHMPLGFLGP (SEQ ID NO:15), SGGPGPAGMKGLPGS (SEQ ID NO: 16), PLGLAG (SEQ ID NO:17), LALGPG (SEQ ID NO:18), KRALGLPG (SEQ ID NO: 19), GGGRR (SEQ ID NO:20), LSGRSDNH (SEQ ID NO:21), PMAKK (SEQ ID NO:22), RQARVVNG (SEQ ID NO:23), HSSKLQL (SEQ ID NO:24) and RRSSYYSG (SEQ ID NO:25).

14. A therapeutic conjugate comprising the fusion protein of claim 1 and a cytotoxic agent.

15. A method of treating cancer in a subject in need thereof, comprising administering to the subject a therapeutically effective amount of the therapeutic conjugate of claim 14.

16. The fusion protein of claim 11, wherein the half-life-extending region is an Fc-binding region or an albumin-binding region (ABR).

17. The therapeutic conjugate of claim 14, wherein the cytotoxic agent is a cytotoxic molecule, peptide, protein or radionuclide.
